SEQUENCE LISTING (77 sequences)

SEQ ID NO: 1 - DNA, 29 bp, Artificial sequence
  Synthetic oligonucleotide.
cggaattcaaggagctagaacagtttgcc

SEQ ID NO: 2 - DNA, 22 bp, Artificial sequence
  Synthetic oligonucleotide.
ctgaaggttctcattgttgtcg

SEQ ID NO: 3 - DNA, 20 bp, Artificial sequence
  Synthetic oligonucleotide.
gatcactcacatcgccaatc

SEQ ID NO: 4 - DNA, 22 bp, Artificial sequence
  Synthetic oligonucleotide.
ctgggaaaggtgtcctgtagcc

SEQ ID NO: 5 - DNA, 25 bp, Artificial sequence
  Synthetic oligonucleotide.
gcgggaagggagaagacactgcgtc

SEQ ID NO: 6 - DNA, 24 bp, Artificial sequence
  Synthetic oligonucleotide.
taggagggccgggttgttactgct

SEQ ID NO: 7 - DNA, 18 bp, Artificial sequence
  Synthetic oligonucleotide.
aggtgcaggctgcctatc

SEQ ID NO: 8 - DNA, 20 bp, Artificial sequence
  Synthetic oligonucleotide.
tggcgtaatcatggtcatag

SEQ ID NO: 9 - DNA, 23 bp, Artificial sequence
  Synthetic oligonucleotide.
gcaacgcaattaatgtgagttag

SEQ ID NO: 10 - DNA, 21 bp, Artificial sequence
  Synthetic oligonucleotide.
ctggatccgctgcattaatga

SEQ ID NO: 11 - DNA, 17 bp, Artificial sequence
  Synthetic oligonucleotide.
ccgagcgcagcgagtca

SEQ ID NO: 12 - DNA, 21 bp, Artificial sequence
  Synthetic oligonucleotide.
gccttatccggtaactatcgt

SEQ ID NO: 13 - DNA, 18 bp, Artificial sequence
  Synthetic oligonucleotide.
gcaccgcctacatacctc

SEQ ID NO: 14 - DNA, 22 bp, Artificial sequence
  Synthetic oligonucleotide.
agttgcctgactccccgtcgtg

SEQ ID NO: 15 - DNA, 20 bp, Artificial sequence
  Synthetic oligonucleotide.
ggagccggtgagcgtgggtc

SEQ ID NO: 16 - DNA, 24 bp, Artificial sequence
  Synthetic oligonucleotide.
ccgatcgttgtcagaagtaagttg

SEQ ID NO: 17 - DNA, 22 bp, Artificial sequence
  Synthetic oligonucleotide.
tcacagaaaagcatcttacgga

SEQ ID NO: 18 - DNA, 24 bp, Artificial sequence
  Synthetic oligonucleotide.
gaaaagtgccacctggtcgacatt

SEQ ID NO: 19 - DNA, 24 bp, Artificial sequence
  Synthetic oligonucleotide.
gggccatttaccgtaagttatgta

SEQ ID NO: 20 - DNA, 19 bp, Artificial sequence
  Synthetic oligonucleotide.
tatcatatgccaagtacgc

SEQ ID NO: 21 - DNA, 22 bp, Artificial sequence
  Synthetic oligonucleotide.
tagatgtactgccaagtaggaa

SEQ ID NO: 22 - DNA, 19 bp, Artificial sequence
  Synthetic oligonucleotide.
tctgactgaccgcgttact

SEQ ID NO: 23 - DNA, 22 bp, Artificial sequence
  Synthetic oligonucleotide.
agaaaagaaacgagccgtcatt

SEQ ID NO: 24 - DNA, 21 bp, Artificial sequence
  Synthetic oligonucleotide.
gggggctgcgaggggaacaaa

SEQ ID NO: 25 - DNA, 21 bp, Artificial sequence
  Synthetic oligonucleotide.
gccgggccgtgctcagcaact

SEQ ID NO: 26 - DNA, 24 bp, Artificial sequence
  Synthetic oligonucleotide.
gcgagccgcagccattgcctttta

SEQ ID NO: 27 - DNA, 23 bp, Artificial sequence
  Synthetic oligonucleotide.
cccagatttcggctccgccagat

SEQ ID NO: 28 - DNA, 23 bp, Artificial sequence
  Synthetic oligonucleotide.
tctttccaccaggcccccggctc

SEQ ID NO: 29 - DNA, 23 bp, Artificial sequence
  Synthetic oligonucleotide.
tgcgggcggacatggggagatcc

SEQ ID NO: 30 - DNA, 23 bp, Artificial sequence
  Synthetic oligonucleotide.
tagagctagactccgggcgatga

SEQ ID NO: 31 - DNA, 23 bp, Artificial sequence
  Synthetic oligonucleotide.
ttgccttaaacaagaccacgaaa

SEQ ID NO: 32 - DNA, 24 bp, Artificial sequence
  Synthetic oligonucleotide.
gcgaactcacacaggcgagaaacc

SEQ ID NO: 33 - DNA, 23 bp, Artificial sequence
  Synthetic oligonucleotide.
tcgcttcctcttcctccgacaca

SEQ ID NO: 34 - DNA, 28 bp, Artificial sequence
  Synthetic oligonucleotide.
tgacctaactcgaggaggagctggaatc

SEQ ID NO: 35 - DNA, 32 bp, Artificial sequence
  Synthetic oligonucleotide.
aagtttgaggcagttaaaattatggctgaagc

SEQ ID NO: 36 - DNA, 20 bp, Artificial sequence
  Synthetic oligonucleotide.
caggtgtttgagggtagctc

SEQ ID NO: 37 - DNA, 20 bp, Artificial sequence
  Synthetic oligonucleotide.
cggttcatcatggtacagtc

SEQ ID NO: 38 - DNA, 24 bp, Artificial sequence
  Synthetic oligonucleotide.
gaagtctggttccttggcaggatg

SEQ ID NO: 39 - DNA, 20 bp, Artificial sequence
  Synthetic oligonucleotide.
actcgatacactggcctagc

SEQ ID NO: 40 - DNA, 24 bp, Artificial sequence
  Synthetic oligonucleotide.
acgagtggcagtttcttcttggga

SEQ ID NO: 41 - DNA, 24 bp, Artificial sequence
  Synthetic oligonucleotide.
tatgactcacttccagggggcact

SEQ ID NO: 42 - DNA, 20 bp, Artificial sequence
  Synthetic oligonucleotide.
accacagtccatgccatcac

SEQ ID NO: 43 - DNA, 20 bp, Artificial sequence
  Synthetic oligonucleotide.
tccaccaccctgttgctgta

SEQ ID NO: 44 - DNA, 26 bp, Artificial sequence
  Synthetic oligonucleotide.
ctgccctgccgggatggcacggaatc

SEQ ID NO: 45 - DNA, 27 bp, Artificial sequence
  Synthetic oligonucleotide.
ttctggccctcaggtcgggtcggcaac

SEQ ID NO: 46 - DNA, 21 bp, Artificial sequence
  Synthetic oligonucleotide.
tggtcactggggacaagggaa

SEQ ID NO: 47 - DNA, 21 bp, Artificial sequence
  Synthetic olignucleotide.
gcaacaacagcaatagagaac

SEQ ID NO: 48 - DNA, 20 bp, Artificial sequence
  Synthetic oligonucleotide.
cacagccctcaccagcagcc

SEQ ID NO: 49 - DNA, 20 bp, Artificial sequence
  Synthetic oligonucleotide.
gactgcctgggctctgctgc

SEQ ID NO: 50 - DNA, 21 bp, Artificial sequence
  Synthetic oligonucleotide.
acacgtccccatctgaaggtg

SEQ ID NO: 51 - DNA, 21 bp, Artificial sequence
  Synthetic oligonucleotide.
cttccttcttcatgccagccc

SEQ ID NO: 52 - DNA, 18 bp, Artificial sequence
  Synthetic oligonucleotide.
cggacatctccccatacg

SEQ ID NO: 53 - DNA, 18 bp, Artificial sequence
  Synthetic oligonucleotide.
aaagggagctggacgcgg

SEQ ID NO: 54 - DNA, 20 bp, Artificial sequence
  Syntehtic oligonucleotide.
gggtgagccatgagccggtg

SEQ ID NO: 55 - DNA, 20 bp, Artificial sequence
  Synthetic oligonucleotide.
catagccgcgccgggatgag

SEQ ID NO: 56 - DNA, 20 bp, Artificial sequence
  Synthetic oligonucleotide.
tggagctgggaggaagcccc

SEQ ID NO: 57 - DNA, 20 bp, Artificial sequence
  Synthetic oligonucleotide.
attgcaaaggggtggggcgg

SEQ ID NO: 58 - DNA, 21 bp, Artificial sequence
  Synthetic oligonucleotide.
tccgctacaatcaaaaaccat

SEQ ID NO: 59 - DNA, 20 bp, Artificial sequence
  Synthetic oligonucleotide.
gctgggtagtggtgggtcta

SEQ ID NO: 60 - DNA, 21 bp, Artificial sequence
  Synthetic oligonucleotide.
cggtgggacttgtgctgctgg

SEQ ID NO: 61 - DNA, 24 bp, Artificial sequence
  Synthetic oligonucleotide.
ctctgaagacgccaggaattccat

SEQ ID NO: 62 - DNA, 20 bp, Artificial sequence
  Synthetic oligonucleotide.
cggggactccacaccccaca

SEQ ID NO: 63 - DNA, 20 bp, Artificial sequence
  Synthetic oligonucleotide.
tgggggccaggtctggtctg

SEQ ID NO: 64 - DNA, 20 bp, Artificial sequence
  Synthetic oligonucleotide.
agaagggcagagcttgggcc

SEQ ID NO: 65 - DNA, 20 bp, Artificial sequence
  Synthetic oligonucleotide.
tgctgcctggccctccaagt

SEQ ID NO: 66 - DNA, 18 bp, Artificial sequence
  Synthetic oligonucleotide.
atgctgtcctgccgtctc

SEQ ID NO: 67 - DNA, 18 bp, Artificial sequence
  Synthetic oligonucleotide.
atgctgtcctgccgtctc

SEQ ID NO: 68 - DNA, 22 bp, Artificial sequence
  Synthetic oligonucleotide.
cttggccaagaactacatctgg

SEQ ID NO: 69 - DNA, 21 bp, Artificial sequence
  Synthetic oligonucleotide.
ggagtagggatgcaccgggaa

SEQ ID NO: 70 - DNA, 20 bp, Artificial sequence
  Synthetic oligonucleotide.
gctgccaggtgcttcccagg

SEQ ID NO: 71 - DNA, 20 bp, Artificial sequence
  Synthetic peptide.
tccagcacaggcaaggcagc

SEQ ID NO: 72 - DNA, 20 bp, Artificial sequence
  Synthetic oligonucleotide.
ccgcagcactcgagcaccaa

SEQ ID NO: 73 - DNA, 20 bp, Artificial sequence
  Synthetic oligonucleotide.
ggcttctttcaccgcccgct

SEQ ID NO: 74 - DNA, 20 bp, Artificial sequence
  Synthetic oligonucleotide.
aaccgtgccacgcgctcaaa

SEQ ID NO: 75 - DNA, 21 bp, Artificial sequence
  Synthetic oligonucleotide.
agggcctaaggcctccagtct

SEQ ID NO: 76 - DNA, 20 bp, Artificial sequence
  Synthetic oligonucleotide.
ggcagccgaacccatctcgg

SEQ ID NO: 77 - DNA, 20 bp, Artificial sequence
  Synthetic oligonucleotide.
agcaggtccgcaaggtgtgc

(19) United States
(12) Patent Application Publication
(10) Pub. No.: US 2012/0135015 A1
(43) Pub. Date: May 31, 2012

(54) Induction of Pancreatic Stem Cells by Transient Overexpression of Reprogramming Factors and PDX1 Selection

(75) Inventors: Hirofumi Noguchi, Okayama (JP); Marlon F. Levy, Dallas, TX (US); Shinichi Matsumoto, Arlington, TX (US)

(73) Assignee: Baylor Research Institute, Dallas, TX (US)

(21) Appl. No.: 13/236,367

(22) Filed: Sep. 19, 2011

Related U.S. Application Data
(60) Provisional application No. 61/387,431, filed on Sep. 28, 2010.

(51) Int. Cl. A61K 35/39; A61P 3/10; C12N 5/10; C12N 15/87; C12Q 1/68

(52) U.S. Cl. 424/184.1; 424/93.21; 435/465; 435/6.12; 435/325; 435/455

(57) ABSTRACT

Methods for generating pancreatic stem cells from a pancreatic tissue of 24-week old mice by transient overexpression of reprogramming factors combined with Pdx1 selection is described herein. The generated cells were designated as iPaS (induced pancreatic stem) cells and exhibit the same morphology as the pancreatic stem cells previously established from young donors without genetic manipulation and express genetic markers of endoderm and pancreatic progenitors. Transplantation of the iPaS cells into nude mice resulted in no teratoma formation. Moreover, iPaS cells were able to differentiate into insulin-producing cells more efficiently than ES cells. In addition, the technology of transient overexpression of reprogramming factors and tissue-specific selection of the present invention may also be useful for the generation of other tissue-specific stem cells.

### CROSS-REFERENCE TO RELATED APPLICATIONS

This patent application is a non-provisional application of U.S. Provisional Patent Application No. 61/387,431 filed on Sep. 28, 2010 and entitled “Induction of Pancreatic Stem Cells by Transient Overexpression of Reprogramming Factors and PDX1 Selection” which is hereby incorporated by reference in its entirety.

### TECHNICAL FIELD OF THE INVENTION

The present invention relates in general to the field of stem cells, and more particularly to the generation of pancreatic stem cells from pancreatic tissue by transient overexpression of reprogramming factors combined with Pdx1 selection.

### STATEMENT OF FEDERALLY FUNDED RESEARCH

None.

### REFERENCE TO A SEQUENCE LISTING

The present application includes a Sequence Listing filed separately as required by 37 CFR 1.821-1.825.

### BACKGROUND OF THE INVENTION

Without limiting the scope of the invention, its background is described in connection induced pluripotent (iPS) stem cell generation.

U.S. Patent Application Publication No. 2008/0233649 (Seaberg et al. 2008) discloses a method for producing isolated clonal stem cell populations from a pancreatic tissue of a mammal, comprising: dissociating all or part of the tissue into single cells, culturing the cells in serum-free media for a time period sufficient that each proliferative pancreatic stem cell has repeatedly divided to produce a corresponding clonal cell population, isolating one of the corresponding clonal cell populations. The clonal pancreatic stem cells express cell markers Pdx-1 and nestin and further express at least one of the cell markers: Sox2, Sox3, Mash1, and Ngn3.

U.S. Patent Application Publication No. 2010/0137202 (Yang, 2010) provides therapeutic compositions and methods for treating a disease, disorder, or injury characterized by a deficiency in the number or biological activity of a cell of interest. The method provides compositions for generating reprogrammed cells or for increasing regeneration in a cell, tissue or organ of interest. The invention describes a method for generating an insulin producing cell in a mammal for the treatment of hyperglycemia, the method comprising: (a) contacting an organ or tissue with a pancreatic transcription factor or fragment thereof comprising a protein transduction domain; and (b) increasing the expression of insulin in a cell of the organ or tissue, thereby generating an insulin producing cell.

### SUMMARY OF THE INVENTION

The present invention describes the generation of pancreatic stem cells from pancreatic tissue by transient overexpression of reprogramming factors combined with Pdx1 selection. In one embodiment the instant invention discloses a composition for islet transplantation comprising one or more induced pancreatic stem (iPaS) cells. The iPaS cells disclosed herein are obtained from differentiated pancreatic ductal cells that are modified into one or more insulin-producing cells by the expression of one or more transcription factors and by an expression of one or more genes selected from the group consisting of Oct3/4, Sox2, Klf4, and c-Myc. In one aspect the transcription factor is Pdx1 and the iPaS cells are generated from a pancreatic tissue of a donor. In another aspect the donor is a human donor, a mouse, a primate or any other vertebrate species. In yet another aspect the composition is used for the treatment of diabetes.

Another embodiment of the present invention provides a method for generating one or more induced pancreatic stem (iPaS) cells from a pancreatic tissue of a vertebrate donor comprising the steps of: (i) digesting the pancreatic tissue from the vertebrate donor, (ii) removing one or more fibroblast cells from the digested tissue cells, (iii) culturing the digested tissue cells without the fibroblast cells in a growth medium, (iv) transfecting the cultured cells with a first plasmid encoding one or more cell marker genes and a promoter, wherein the cell marker genes are selected from the group consisting of Oct3/4, Sox2, Klf4, and c-Myc, (v) transfecting the cultured cells with a second plasmid encoding one or more transcription factors, wherein the transcription factor comprises Pdx1, and (vi) harvesting one or more colonies of iPaS cells following the transfection of the first and the second plasmid.

The method described hereinabove further comprising the steps of performing a polymerase chain reaction (PCR) analysis on the transfected cells to determine a plasmid integration and an expression of one or more cell marker genes and performing an immunoassay or any other suitable assay to determine a level of insulin produced by the generated iPaS cells. The present invention specifically discloses an induced pancreatic stem (iPaS) cell made by the method above.

In yet another embodiment the present invention relates to a method of treating diabetes in a patient comprising the steps of: identifying the patient in need of treatment against the diabetes, infusing a therapeutically effective amount of an islet transplantation composition into a liver of the patient through a catheter, wherein the islet transplantation composition comprises one or more induced pancreatic stem (iPaS) cells, and administering an optional immunosuppressant to the patient to prevent a rejection of the one or more infused islets. In one aspect the iPaS cells differentiates into one or more insulin-producing cells under an influence of one or more transcription factors. In one aspect the transcription factor is Pdx1. In another aspect the iPaS cells express one or more cell markers selected from the group consisting of Oct3/4, Sox2, Klf4, and c-Myc. In another aspect the iPaS cells are generated from a pancreatic tissue of a donor, wherein the donor is a human donor, a mouse, a primate or any other vertebrate species. In yet another aspect the method further comprises the step of measuring a glucose level, an insulin level or both in the patient at one or more definite intervals post transplantation.

The instant invention also describes an induced pluripotent stem (iPS) cell colony, wherein the iPS cell colony is made from a tissue of a donor by transfection with one or more plasmids encoding one or more transcription factors, cell marker genes or both. In one aspect the donor comprises a human donor, a mouse, a primate or any other vertebrate species. In another aspect the tissue comprises a pancreatic tissue, a kidney tissue, a liver tissue, a heart tissue or a splenic tissue.

In another embodiment the present invention describes a method for generating one or more induced pluripotent stem (iPS) cells ex vivo from a pancreatic tissue of a donor comprising the steps of: (i) digesting the donor tissue, (ii) culturing the digested tissue cells in a growth medium, (iii) transfecting the cultured cells with one or more plasmids encoding one or more cell marker genes and a promoter, a transcription factor or both, and (iv) harvesting one or more colonies of iPS cells following the transfection of the plasmid. The iPS cell generating method further comprising the steps of: performing an optional step of removing one or more fibroblast cells from the digested tissue cells and performing a PCR analysis of the transfected cells to determine a plasmid integration and an expression of one or more cell marker genes. In one aspect the donor comprises a human donor, a mouse, a primate or any other vertebrate species.

In another aspect the tissue comprises a pancreatic tissue, a kidney tissue, a liver tissue, a heart tissue or a splenic tissue. In a specific aspect the tissue is a pancreatic tissue. In yet another aspect the cell marker genes are selected from the group consisting of Oct3/4, Sox2, Klf4, and c-Myc and the transcription factor is Pdx1. Finally, the present invention discloses an induced pluripotent stem (iPS) cell generated by the method described hereinabove.

### BRIEF DESCRIPTION OF THE DRAWINGS

For a more complete understanding of the features and advantages of the present invention, reference is now made to the detailed description of the invention along with the accompanying figures and in which:

FIGS. 1A-1D show the generation of iPaS 4F cells from mouse pancreatic tissue: FIG. 1A expression plasmid for iPaS cell generation. The four cDNAs encoding Oct3/4, Sox2, Klf4, and c-Myc were connected in this order with the 2A peptide and inserted into the plasmid containing the CAG promoter. IRES and hygromycin resistant genes were also inserted into the plasmid. Thick lines (O-1, O-2, K, and 1 to 11) indicate amplified regions used in (D) to detect plasmid integration into the genome. The locations of the CAG promoter, the ampicillin-resistant gene (AmpR), and the polyadenylation signal (pA) are also shown, FIG. 1B time schedules for induction of iPaS 4F cells with the plasmid. Open arrowheads indicate the timing of cell seed, passage, and colony pickup. Solid arrow heads indicate the timing of transfection. Selection by hygromycin was performed from immediately after the last transfection (afternoon of day 7) to just before passage, FIG. 1C morphology of HN#13 cells, mouse pancreatic tissue, iPaS 4F-1, iPaS 4F-5, and iFL cells. Scale bars=200 μm, FIG. 1D detection of plasmid integration by PCR. Genomic DNA from pancreatic tissue (Pa), iPaS 4F-1, iPaS 4F-5, iFL, HN#13 (H), and ES (E) cells were amplified by PCR to generate the amplified regions indicated in (A). An expression plasmid was used as a positive control (P1). In PCR for O-1, O-2, and K, bands derived from the endogenous (endo) genes are shown with open arrowheads, whereas those from integrated plasmids (Tg) are shown with solid arrowheads;

FIGS. 2A-2E show the characterization of iPaS 4F cells: FIG. 2A RT-PCR analysis of ES cell marker genes in iPaS 4F cells. Total RNAs isolated from pancreatic tissue (Pa), iPaS 4F-1, iPaS 4F-5, iFL, HN#13 (H), and ES (E) cells were analyzed with RT-PCR, FIG. 2B schematic representation of stepwise differentiation of ES cells to insulin-producing cells. Cells of the definitive endoderm (DE) express Foxa2 and Sox17; cells of the gut tube endoderm (GTE) express Hnf1β and Hnf 4α; cells of pancreatic progenitors (PP) express Pdx1 and Hnf6; and insulin-producing cells (IPC) express insulin, Glut4, and glucokinase (GK), FIG. 2C RT-PCR analysis of endodermal/pancreatic cell marker genes in iPaS 4F cells. iPaS 4F-1, iPaS 4F-5, iFL, and HN#13 (H) were analyzed by RT-PCR. Differentiated cells (DE, GTE, PP) derived from ES cells by the stepwise protocol were used as a positive control, FIG. 2D growth curves of HN#13 cells and iPaS 4F-1 (PDL50 and 300), FIG. 2E teratoma/tumorigenic Assay. 1×10⁷ of iPaS 4F-1 cells were inoculated into one of the thighs of nude mice. As a positive control, we transplanted 1×10⁷ ES cells into the other thighs of the nude mice;

FIGS. 3A-3D show differentiation of iPaS 4F Cells into insulin-producing cells: FIG. 3A immunostaining of iPaS 4F-1 cells (Pdx1) and insulin-producing cells derived from iPaS 4F-1 cells (insulin, C-peptide). A mouse pancreas was used as a positive control. Insulin staining of iFL cells treated with the stepwise protocol was also performed. Scale bars=100 μm, FIG. 3B RT-PCR analysis of pancreatic β cell marker genes in differentiated iPaS 4F cells. Differentiated cells derived from iPaS 4F-1 cells by stage 1-5 or 4-5, and derived from ES cells by stage 1-5 or 4-5 were analyzed with RT-PCR. Stage 1-5 treated iFL cells were also analyzed with RT-PCR. Isolated islets were used as a positive control, FIG. 3C quantitative RT-PCR analysis of insulin genes in differentiated iPaS 4F cells. Differentiated cells derived from iPaS 4F-1 cells by stage 1-5 or 4-5, and derived from ES cells by stage 1-5 or 4-5 were analyzed with quantitative RT-PCR. Isolated islets were used as a positive control, FIG. 3D insulin release assay. Differentiated iPaS 4F-1 cells by stage 4-5 and derived from ES cells by stage 4-5 were stimulated with 2.8 and 20 mM D-glucose, and the amount of insulin released to culture supernatant was analyzed by ELISA;

FIGS. 4A-4D show the generation of iPaS 4FP Cells by Expression Plasmid and Pdx1 selection: FIG. 4A selection plasmid for iPaS cell generation. The Cre gene in a Pdx1-Cre plasmid (Addgene: Plasmid 15021 (DM#258)) was replaced with a bleomycin resistance gene that was derived from pIRES-bleo (Clontech). Thick lines (5, 6) indicate amplified regions used in (D) since the plasmid has an AmpR gene. The locations of the Pdx1 promoter, bleomycin resistant gene (BleoR), the ampicillin-resistant gene (AmpR), and the polyadenylation signal (pA) are shown, FIG. 4B time schedules for induction and selection of iPaS cells with the plasmid. Open arrowheads indicate the timing of cell seed, passage, and colony pickup. Solid arrowheads indicate the timing of transfection. Selections by hygromycin and bleomycin were performed from immediately after the last transfection (afternoon of day 7) to just before passage; FIG. 4C morphology of iPaS 4FP-1 to 6 cells. Scale bars=200 μm, FIG. 4D detection of plasmid integration by PCR. Genomic DNA from pancreatic tissue (Pa), iPaS 4FP-1 to 6, HN#13 (H), and ES (E) cells were amplified by PCR with the primers indicated in FIG. 1A (O-1, O2, K, and 1 to 11) and 4A (5, 6). The expression plasmid was used as a positive control (P1). In PCR for O-1, O-2, and K, bands derived from the endogenous (endo) genes are shown with open arrowheads, whereas those from integrated plasmids (Tg) are shown with solid arrowheads;

FIGS. 5A-5C show the characterization of iPaS 4FP cells: FIG. 5A RT-PCR analysis of ES cell marker genes in iPaS 4FP cells. Total RNAs isolated from pancreatic tissue (Pa), iPaS 4FP-1, -2, -3, -5, HN#13 (H), and ES (E) cells were analyzed by RT-PCR, FIG. 5B RT-PCR analysis of endodermal/pancreatic cell marker genes in iPaS 4FP cells. iPaS 4FP-1, -2, -3, -5, and HN#13 (H) were analyzed by RT-PCR. Differentiated cells (DE, GTE, PP), derived from ES cells by the stepwise protocol, were used as a positive control, FIG. 5C teratoma/tumorigenic Assay. 1×10⁷ of iPaS 4FP-2 cells were inoculated into one side of the two thighs of nude mice. As a positive control, we transplanted 1×10⁷ ES cells into the other thigh of the nude mice;

FIGS. 6A-6D show immunostaining of iPaS 4FP Cells: FIG. 6A immunostaining of iPaS 4FP-2 cells and insulin-producing cells derived from iPaS 4FP-2 cells (insulin, C-peptide). Scale bars=100 μm, FIG. 6B RT-PCR analysis of pancreatic β cell marker genes in differentiated iPaS cells. Differentiated cells derived from iPaS 4FP-1, -2, -3, and -5 cells by stage 4-5, and undifferentiated iPaS 4FP-2 cells were analyzed with RT-PCR. Isolated islets were used as a positive control, FIG. 6C quantitative RT-PCR analysis of insulin genes in differentiated iPaS 4FP cells. Differentiated cells derived from iPaS 4FP-1, -2, -3, and -5 cells by stage 4-5 were analyzed with quantitative RT-PCR. Isolated islets were used as a positive control, FIG. 6D insulin release assay. Differentiated iPaS 4FP-1, -2, -3, and -5 cells by stage 4-5 were stimulated with 2.8 and 20 mM D-glucose, and the amount of insulin released to culture supernatant was analyzed by ELISA; and

FIG. 7 shows the immunostaining of iPaS 4FP cells immunostaining of insulin-producing cells derived from iPaS 4FP-2 cells (insulin, glucagon). A mouse pancreas was used as a positive control (insulin, glucagon). Scale bars=100 μm

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

The term “diabetes” as described in embodiments of the present invention refers to the chronic disease characterized by relative or absolute deficiency of insulin that results in glucose intolerance. The term “diabetes” is also intended to include those individuals with hyperglycemia, including chronic hyperglycemia, hyperinsulinemia, impaired glucose homeostasis or tolerance, and insulin resistance.

The term “insulin” as used herein shall be interpreted to encompass insulin analogs, natural extracted human insulin, recombinantly produced human insulin, insulin extracted from bovine and/or porcine sources, recombinantly produced porcine and bovine insulin and mixtures of any of these insulin products. The term is intended to encompass the polypeptide normally used in the treatment of diabetics in a substantially purified form but encompasses the use of the term in its commercially available pharmaceutical form, which includes additional excipients. The insulin is preferably recombinantly produced and may be dehydrated (completely dried) or in solution.

The term “islet cell (s)” as used throughout the specification is a general term to describe the clumps of cells within the pancreas known as islets, e.g., islets of Langerhans. Islets of Langerhans contain several cell types that include, e.g., β-cells (which make insulin), α-cells (which produce glucagons), γ-cells (which make somatostatin), F cells (which produce pancreatic polypeptide), enterochromaffin cells (which produce serotonin), PP cells and D1 cells. The term “stem cell” is an art recognized term that refers to cells having the ability to divide for indefinite periods in culture and to give rise to specialized cells. Included within this term are, for example, totipotent, pluripotent, multipotent, and unipotent stem cells, e.g., neuronal, liver, muscle, and hematopoietic stem cells.

As used herein, the term “pluripotent stem cell” refers to a cell that has the ability to self replicate for indefinite periods and can give rise to may cell types under the right conditions, particularly, the cell types that derived from all three embryonic germ layers: mesoderm, endoderm, and ectoderm. As used herein, the term “feeder cells” refers to cells of one tissue type that are co-cultured with cells of another tissue type, to provide an environment in which cells of the second tissue type may grow. The feeder cells are optionally from a different species as the cells they are supporting.

The term “gene” is used to refer to a functional protein, polypeptide or peptide-encoding unit. As will be understood by those in the art, this functional term includes both genomic sequences, cDNA sequences, or fragments or combinations thereof, as well as gene products, including those that may have been altered by the hand of man. Purified genes, nucleic acids, protein and the like are used to refer to these entities when identified and separated from at least one contaminating nucleic acid or protein with which it is ordinarily associated.

The term “plasmid” for purposes of the present invention includes any type of replication vector which has the capability of having a non-endogenous DNA fragment inserted into it. Procedures for the construction of plasmids include those described in Maniatis et al., Molecular Cloning, A Laboratory Manual, 2d, Cold Spring Harbor Laboratory Press (1989).

As used herein, the term “promoter” is defined as a DNA sequence recognized by the synthetic machinery of the cell, or introduced synthetic machinery, required to initiate the specific transcription of a gene.

The term “transcription factor” is intended to encompass all proteins which recognize and specifically bind to cis-regulatory DNA sequence elements of a gene, wherein the binding of those transcription factors to those cis-regulatory DNA sequence elements has the effect of altering the transcriptional expression of that specific gene.

As used herein, the term “transfection” means the introduction of DNA, RNA, other genetic material, protein or organelle into a target cell.

The term “vertebrate” as used herein includes species of fish, amphibians, reptiles, birds and mammals that possess a Hepp gene or equivalent.

As used herein, the term “polymerase chain reaction” (PCR) refers to the method of K. B. Mullis U.S. Pat. Nos. 4,683,195, 4,683,202, and 4,965,188, hereby incorporated by reference, which describe a method for increasing the concentration of a segment of a target sequence in a mixture of genomic DNA without cloning or purification. This process for amplifying the target sequence consists of introducing a large excess of two oligonucleotide primers to the DNA mixture containing the desired target sequence, followed by a precise sequence of thermal cycling in the presence of a DNA polymerase. The two primers are complementary to their respective strands of the double stranded target sequence. To effect amplification, the mixture is denatured and the primers then annealed to their complementary sequences within the target molecule. Following annealing, the primers are extended with a polymerase so as to form a new pair of complementary strands. The steps of denaturation, primer annealing and polymerase extension can be repeated many times (i.e., denaturation, annealing and extension constitute one “cycle”; there can be numerous “cycles”) to obtain a high concentration of an amplified segment of the desired target sequence. The length of the amplified segment of the desired target sequence is determined by the relative positions of the primers with respect to each other, and therefore, this length is a controllable parameter. By virtue of the repeating aspect of the process, the method is referred to as the “polymerase chain reaction” (hereinafter “PCR”). Because the desired amplified segments of the target sequence become the predominant sequences (in terms of concentration) in the mixture, they are said to be “PCR amplified”. With PCR, it is possible to amplify a single copy of a specific target sequence in genomic DNA to a level detectable by several different methodologies (e.g., hybridization with a labeled probe; incorporation of biotinylated primers followed by avidin-enzyme conjugate detection; incorporation of 32P-labeled deoxynucleotide triphosphates, such as DCTP or DATP, into the amplified segment). In addition to genomic DNA, any oligonucleotide sequence can be amplified with the appropriate set of primer molecules. In particular the amplified segments created by the PCR process itself are, themselves, efficient templates for subsequent PCR amplifications.

As used herein, the term “in vivo” refers to being inside the body. The term “in vitro” used as used in the present application is to be understood as indicating an operation carried out in a non-living system.

As used herein, the term “treatment” or “treating” refers to any administration of a compound of the present invention and includes (1) inhibiting the disease in an animal that is experiencing or displaying the pathology or symptomatology of the diseased (i.e., arresting further development of the pathology and/or symptomatology), or (2) ameliorating the disease in an animal that is experiencing or displaying the pathology or symptomatology of the diseased (i.e., reversing the pathology and/or symptomatology).

The present invention describes the generation of induced pluripotent stem (iPS) cells. The inventors generated pancreatic stem cells from pancreatic tissue of mice by transient overexpression of reprogramming factors combined with Pdx1 selection. The generated cells exhibited the same morphology as the pancreatic stem cells that were previously established by the inventors from young donors without genetic manipulation and express genetic markers of endoderm and pancreatic progenitors. The iPaS cells generated herein were able to differentiate into insulin-producing cells more efficiently than ES cells.

Diabetes mellitus is a devastating disease. The World Health Organization (WHO) expects that the number of diabetic patients to increase to 300 million by the year 2025. It is now well established that the risk of diabetic complications is dependent on the degree of glycemic control in diabetic patients and that tight glycemic control achieved with intensive insulin regimens can reduce the risk of developing or progressing retinopathy, nephropathy or neuropathy in patients with all types of diabetes. However, intensive glycemic control with insulin therapy is associated with an increased incidence of hypoglycemia, which is the major barrier to the implementation of intensive treatment from the perspective of both physicians and patients. Pancreas and pancreatic islet transplantation can achieve insulin independence in patients with type 1 diabetes (Shapiro et al. 2000). However, the clinical benefit of these protocols can be provided only to a small minority of patients and they have the risks associated with the use of immunosuppressant drugs. Nonetheless, the promising results afforded by pancreas transplantation and, especially, isolated islets, coupled with the shortage of cadaver pancreata relative to the potential demand, have lent a strong impetus to the search for new sources of insulin-producing cells.

Adult tissue-specific stem/progenitor cells could be one of the alternative sources for the treatment of diabetes. Islet neogenesis, the budding of new islets from pancreatic stem/progenitor cells located in or near ducts, has long been assumed to be an active process in the postnatal pancreas. Several in vitro studies have shown that insulin-producing cells can be generated from adult pancreatic ductal tissues (Bonner-Weir, et al., 2000; Heremans, et al., 2002; Gao, et al. 2003). The assessment of eighty-three human islet grafts transplanted using the Edmonton Protocol since 1999 (Street, et al., 2004) showed that a significant positive correlation was observed between the number of islet progenitor (ductal-epithelial) cells transplanted and long-term metabolic success, as assessed by an intravenous glucose tolerance test at approximately two years post-transplantation. Therefore, pancreatic stem/progenitor cells could become one of the new sources of insulin-producing cells. One of the most difficult and yet unsolved issues is how to isolate pancreatic stem cells, which have self-renewal capacity. The present inventors and other groups established mouse pancreatic stem cell lines using specific culture conditions (Yamamoto et al., 2006; Noguchi et al., 2009). One of our established pancreatic stem cell lines, HN#13, from the pancreatic tissue of an eight-week-old mouse without genetic manipulation could be maintained by repeated passages for more than one year without growth inhibition in a specific culture condition. HN#13 cells do not have tumorigenic properties, and do have a normal chromosome (Noguchi et al., 2009). The cells express the pancreatic and duodenal homeobox factor-1 (Pdx-1), also known as IDX-1/STF-1/IPF1, one of the transcription factors of β cell lineage. However, it is not yet able to isolate and culture mouse pancreatic stem cells from older donors or pancreatic stem cells from human pancreatic tissue.

Induced pluripotent stem (iPS) cells, which were generated from adult fibroblasts or other somatic cells, are also an alternative source for the treatment of diabetes. Initial iPS cells have been generated from mouse and human somatic cells by introducing Oct3/4 and Sox2 with either 1) Klf4 and c-Myc or 2) Nanog and Lin28 using retroviruses (Takahashi et al., 2006; Takahashi et al., 2007; Yu et al., 2007; Lowry et al., 2008; Park et al., 2008). Mouse and human iPS cells are similar to embryonic stem (ES) cells in morphology, gene expression, epigenetic status and in vitro differentiation. Furthermore, mouse iPS cells give rise to adult chimeras and show competence for germline transmission (Maherali et al., 2007; Okita et al., 2007; Wernig et al., 2007). This technical breakthrough has significant implications for overcoming the ethical issues associate with ES cell derivation from embryos. However, retroviral integration of the transcription factors may activate or inactivate host genes, resulting in tumorigenicity, as was the case in some patients who underwent gene therapy. The generation of mouse iPS cells by repeated transfection of plasmids expressing Oct3/4, Sox2, Klf4 and c-Myc (Okita et al., 2008) and by using nonintegrating adenoviruses transiently expressing the four factors (Stadtfeld et al., 2008) has recently been reported. Moreover, the generation of human iPS cells without genomic integration of exogenous reprogramming factors by plasmids expressing OCT3/4, SOX2, KLF4, c-MYC, NANOG, LIN28, and SV40LT (Yu et al., 2009) has been shown. These reports provide strong evidence that insertional mutagenesis is not required for in vitro reprogramming. The production of iPS cells without viral integration addresses a critical safety concern for potential use of iPS cells in regenerative medicine. However, iPS cells still have some issues, including teratoma formation after transplantation of differentiated cells derived from iPS cells because of contamination of undifferentiated cells.

The present invention describes the generation of pancreatic stem cells (induced pancreatic stem cells; iPaS cells) from mouse pancreatic tissue by transient overexpression of reprogramming factors and Pdx1 selection. These cells have no teratoma formation and are able to differentiate into insulin-producing cells more efficiently than ES cells.

Mice and Cell Culture: Mouse studies were approved by the Baylor Institutional Animal Care and Use Committee (IACUC). Newborn (0-week-old), 8-week-old, and 24-week-old C57/BL6 mice (CREA) were used for primary pancreatic tissue preparations. Mouse pancreata were digested with 2 ml cold M199 medium containing 2 mg/ml collagenase (Roche Boehringer Mannheim). The digested tissues were cultured in Dulbecco's modified Eagle's medium (DMEM; Invitrogen) with 10-20% fetal bovine serum (FBS; BIO-WEST). For the establishment of pancreatic stem cells without genetic manipulation from primary pancreatic tissue, fibroblast-like cells were removed mechanically with a rubber scrapper and the duct-like cells (cobblestone morphology) were cultured in DMEM with 20% FBS and then inoculated into 96-well plates and cloned by limiting dilution (Noguchi et al., 2009).

Mouse ES cells (ATCC) and iPaS cells were maintained in complete ES cell media w/15% FBS (Millipore) on feeder layers of mitomycin C-treated STO cells, as previously described (Takahashi et al., 2006). ES cells were passaged every 3 days and iPaS cells were passaged every 5 days.

Plasmid Construction: To generate the OSKM plasmid, the four cDNAs encoding Oct3/4, Sox2, Klf4, and c-Myc were connected in this order with the 2A peptide and inserted into a plasmid containing the CAG promoter (Niwa et al., 1991). Genes of internal ribosome entry site (IRES) and hygromycin resistance derived from SSR#69 (Noguchi et al., 2002) were introduced into the OSKM plasmid. To generate the pPdx1-BleoR plasmid, the Cre gene in Pdx1-Cre plasmid (Addgene: Plasmid 15021 (DM#258)) was replaced with the bleomycin resistant gene, derived from pIRES-bleo (Clontech).

DNA-PCR: DNA was extracted from cells using the AllPrep DNA/RNA Mini Kit (QIAGEN). Polymerization reactions were performed in a Perkin-Elmer 9700 Thermocycler with 3 μl cDNA (20 ng DNA equivalents), 160 μmol/l cold dNTPs, 10 pmol appropriate oligonucleotide primers, 1.5 mmol/l MgCl2, and 5 units AmpliTaq Gold DNA polymerase (Perkin-Elmer, Norwalk, Conn.) in 1X PCR buffer. The oligonucleotide primers are shown in Table 1. The thermal cycle profile used a ten-minute denaturing step at 94° C. followed by amplification cycles (one minute denaturation at 94° C., one minute annealing at 57-62° C., and one minute extension at 72° C.) with a final extension step of ten minutes at 72° C.

TABLE 1
List of oligonucleotide primers.

| ID | Name | Sequence |
|---|---|---|
| SEQ ID NO: 1 | pr-CX-O-1-s | CGG AAT TCA AGG AGC TAG AAC AGT TTG CC |
| SEQ ID NO: 2 | pr-CX-O-1-as | CTG AAG GTT CTC ATT GTT GTC G |
| SEQ ID NO: 3 | pr-CX-O-2-s | GAT CAC TCA CAT CGC CAA TC |
| SEQ ID NO: 4 | pr-CX-O-2-as | CTG GGA AAG GTG TCC TGT AGC C |
| SEQ ID NO: 5 | pr-CX-K-s | GCG GGA AGG GAG AAG ACA CTG CGT C |
| SEQ ID NO: 6 | pr-CX-K-as | TAG GAG GGC CGG GTT GTT ACT GCT |
| SEQ ID NO: 7 | pr-CX-1-s | AGG TGC AGG CTG CCT ATC |
| SEQ ID NO: 7 | pr-CX-1-as | TTA GCC AGA AGT CAG ATG CTC |
| SEQ ID NO: 8 | pr-CX-2-s | TGG CGT AAT CAT GGT CAT AG |
| SEQ ID NO: 9 | pr-CX-2-as | GCA ACG CAA TTA ATG TGA GTT AG |
| SEQ ID NO: 10 | pr-CX-3-s | CTG GAT CCG CTG CAT TAA TGA |
| SEQ ID NO: 11 | pr-CX-3-as | CCG AGC GCA GCG AGT CA |
| SEQ ID NO: 12 | pr-CX-4-s | GCC TTA TCC GGT AAC TAT CGT |
| SEQ ID NO: 13 | pr-CX-4-as | GCA CCG CCT ACA TAC CTC |
| SEQ ID NO: 14 | pr-CX-5-s | AGT TGC CTG ACT CCC CGT CGT G |
| SEQ ID NO: 15 | pr-CX-5-as | GGA GCC GGT GAG CGT GGG TC |
| SEQ ID NO: 16 | pr-CX-6-s | CCG ATC GTT GTC AGA AGT AAG TTG |
| SEQ ID NO: 17 | pr-CX-6-as | TCA CAG AAA AGC ATC TTA CGG A |
| SEQ ID NO: 18 | pr-CX-7-s | GAA AAG TGC CAC CTG GTC GAC ATT |
| SEQ ID NO: 19 | pr-CX-7-as | GGG CCA TTT ACC GTA AGT TAT GTA |
| SEQ ID NO: 20 | pr-CX-8-s | TAT CAT ATG CCA AGT ACG C |
| SEQ ID NO: 21 | pr-CX-8-as | TAG ATG TAC TGC CAA GTA GGA A |
| SEQ ID NO: 22 | pr-CX-9-s | TCT GAC TGA CCG CGT TAC T |
| SEQ ID NO: 23 | pr-CX-9-as | AGA AAA GAA ACG AGC CGT CAT T |
| SEQ ID NO: 24 | pr-CX-10-s | GGG GGC TGC GAG GGG AAC AAA |
| SEQ ID NO: 25 | pr-CX-10-as | GCC GGG CCG TGC TCA GCA ACT |
| SEQ ID NO: 26 | pr-CX-11-s | GCG AGC CGC AGC CAT TGC CTT TTA |
| SEQ ID NO: 27 | pr-CX-11-as | CCC AGA TTT CGG CTC CGC CAG AT |
| SEQ ID NO: 28 | Oct3/4-s | TCT TTC CAC CAG GCC CCC GGC TC |
| SEQ ID NO: 29 | Oct3/4-as | TGC GGG CGG ACA TGG GGA GAT CC |
| SEQ ID NO: 30 | Sox2-s | TAG AGC TAG ACT CCG GGC GAT GA |
| SEQ ID NO: 31 | Sox2-as | TTG CCT TAA ACA AGA CCA CGA AA |
| SEQ ID NO: 32 | K1f4-s | GCG AAC TCA CAC AGG CGA GAA ACC |
| SEQ ID NO: 33 | K1f4-as | TCG CTT CCT CTT CCT CCG ACA CA |
| SEQ ID NO: 34 | c-Myc-s | TGA CCT AAC TCG AGG AGG AGC TGG AAT C |
| SEQ ID NO: 35 | c-Myc-as | AAG TTT GAG GCA GTT AAA ATT ATG GCT GAA GC |
| SEQ ID NO: 36 | Nanog-s | CAG GTG TTT GAG GGT AGC TC |
| SEQ ID NO: 37 | Nanog-as | CGG TTC ATC ATG GTA CAG TC |
| SEQ ID NO: 38 | Esg1-s | GAA GTC TGG TTC CTT GGC AGG ATG |
| SEQ ID NO: 39 | Esg1-as | ACT CGA TAC ACT GGC CTA GC |
| SEQ ID NO: 40 | Rex1-s | ACG AGT GGC AGT TTC TTC TTG GGA |
| SEQ ID NO: 41 | Rex1-as | TAT GAC TCA CTT CCA GGG GGC ACT |
| SEQ ID NO: 42 | GAPDH-s | ACC ACA GTC CAT GCC ATC AC |
| SEQ ID NO: 43 | GAPDH-as | TCC ACC ACC CTG TTG CTG TA |
| SEQ ID NO: 44 | Sox17-s | CTG CCC TGC CGG GAT GGC ACG GAA TC |
| SEQ ID NO: 45 | Sox17-as | TTC TGG CCC TCA GGT CGG GTC GGC AAC |
| SEQ ID NO: 46 | Foxa2-s | TGG TCA CTG GGG ACA AGG GAA |
| SEQ ID NO: 47 | Foxa2-as | GCA ACA ACA GCA ATA GAG AAC |
| SEQ ID NO: 48 | HNF 1b-s | CAC AGC CCT CAC CAG CAG CC |
| SEQ ID NO: 49 | HNF 1b-as | GAC TGC CTG GGC TCT GCT GC |
| SEQ ID NO: 50 | HNF 4a-s | ACA CGT CCC CAT CTG AAG GTG |
| SEQ ID NO: 51 | HNF 4a-as | CTT CCT TCT TCA TGC CAG CCC |
| SEQ ID NO: 52 | PDX-1-s | CGG ACA TCT CCC CAT ACG |
| SEQ ID NO: 53 | PDX-1-as | AAA GGG AGC TGG ACG CGG |
| SEQ ID NO: 54 | HNF 6-s | GGG TGA GCC ATG AGC CGG TG |
| SEQ ID NO: 55 | HNF 6-as | CAT AGC CGC GCC GGG ATG AG |
| SEQ ID NO: 56 | Insulin1-s | TGG AGC TGG GAG GAA GCC CC |
| SEQ ID NO: 57 | Insulin1-as | ATT GCA AAG GGG TGG GGC GG |
| SEQ ID NO: 58 | Insulin2-s | TCC GCT ACA ATC AAA AAC CAT |
| SEQ ID NO: 59 | Insulin2-as | GCT GGG TAG TGG TGG GTC TA |
| SEQ ID NO: 60 | Glut2-s | CGG TGG GAC TTG TGC TGC TGG |
| SEQ ID NO: 61 | Glut2-as | CTC TGA AGA CGC CAG GAA TTC CAT |
| SEQ ID NO: 62 | Glucokinase-s | CGG GGA CTC CAC ACC CCA CA |
| SEQ ID NO: 63 | Glucokinase-as | TGG GGG CCA GGT CTG GTC TG |
| SEQ ID NO: 64 | Glucagon-s | AGA AGG GCA GAG CTT GGG CC |
| SEQ ID NO: 65 | Glucagon-as | TGC TGC CTG GCC CTC CAA GT |
| SEQ ID NO: 66 | Somatostatin-s | ATG CTG TCC TGC CGT CTC |
| SEQ ID NO: 67 | Somatostatin-as | TTC TCT GTC TGG TTG GGC TC |
| SEQ ID NO: 68 | NeuroD-s | CTT GGC CAA GAA CTA CAT CTG G |
| SEQ ID NO: 69 | NeuroD-as | GGA GTA GGG ATG CAC CGG GAA |
| SEQ ID NO: 70 | Pax4-s | GCT GCC AGG TGC TTC CCA GG |
| SEQ ID NO: 71 | Pax4-as | TCC AGC ACA GGC AAG GCA GC |
| SEQ ID NO: 72 | Pax6-s | CCG CAG CAC TCG AGC ACC AA |
| SEQ ID NO: 73 | Pax6-as | GGC TTC TTT CAC CGC CCG CT |
| SEQ ID NO: 74 | Nkx2.2-s | AAC CGT GCC ACG CGC TCA AA |
| SEQ ID NO: 75 | Nkx2.2-as | AGG GCC TAA GGC CTC CAG TCT |
| SEQ ID NO: 76 | Is1-1-s | GGC AGC CGA ACC CAT CTC GG |
| SEQ ID NO: 77 | Is1-1-as | AGC AGG TCC GCA AGG TGT GC |

RT-PCR: Total RNA was extracted from cells using the AllPrep DNA/RNA Mini Kit or RNeasy Mini Kit (QIAGEN). After quantifying the RNA by spectrophotometry, 2.5 μg of RNA were heated at 85° C. for three minutes and then reverse-transcribed into cDNA in a 25 μl solution containing 200 units of Superscript II RNase H-RT (Invitrogen), 50 ng random hexamers (Invitrogen), 160 μmol/l dNTP, and 10 nmol/l dithiothreitol. The reaction consisted of ten minutes at 25° C., sixty minutes at 42° C., and ten minutes at 95° C. Polymerization reactions were performed, as shown in the DNA-PCR section. The oligonucleotide primers are shown in Table 1.

Cell induction and differentiation: Directed differentiation was conducted, as described (D'Amour et al., 2006; Kroon et al., 2008), with minor modifications. In stage 1, cells were treated with 25 ng/ml of Wnt3a and 100 ng/ml of activin A (R&D Systems) in RPMI (Invitrogen) for 1 day, followed by treatment with 100 ng/ml of activin A in RPMI+0.2% FBS for 2 days. In stage 2, the cells were treated with 50 ng/ml of FGF10 (R&D Systems) and 0.25 μM of KAAD-cyclopamine (Toronto Research Chemicals) in RPMI+2% FBS for 3 days. In stage 3, the cells were treated with 50 ng/ml of FGF10, 0.25 μM of KAAD-cyclopamine, and 2 μM of all-trans retinoic acid (Sigma) in DMEM+1% (vol/vol) B27 supplement (Invitrogen) for 3 days. In stage 4, the cells were treated with 1 μM of DAPT (Sigma) and 50 ng/ml of exendin-4 (Sigma) in DMEM+1% (vol/vol) B27 supplement for 3 days. In stage 5, the cells were then treated with 50 ng/ml of exendin-4, 50 ng/ml of IGF-1 (Sigma), and 50 ng/ml of HGF (R&D Systems) in CMRL (Invitrogen)+1% (vol/vol) B27 supplement for 3-6 days.

Quantitative PCR: Quantification of insulin mRNA levels was carried using the TaqMan real-time PCR system, according to the manufacturer's instructions (Applied Biosystems, Foster City, Calif., USA). PCR was performed for forty cycles, including two minutes at 50° C. and ten minutes at 95° C. as initial steps. In each cycle, denaturation was achieved for fifteen seconds at 95° C. and annealing/extension was achieved for one minute at 60° C. PCR was carried out in 20 μl of solution using cDNAs synthesized from 1.11 ng of total RNA. Standard curves were obtained using cDNAs generated from total RNA isolated from primary mouse islets. For each sample, the expression of insulin was normalized by dividing by the β-actin expression level. Mouse insulin-1, mouse insulin -2 and β-actin primers are commercially available (Assays-on-Demand Gene Expression Products; Applied Biosystems).

Teratoma/Tumorigenic Assay: 1×10⁷ of iPaS cells were inoculated into one thigh each of nude mice. As a positive control, the inventors transplanted 1×10⁷ ES cells into the other thighs of the nude mice.

Immunostaining: Cells were fixed with 4% paraformaldehyde in PBS buffer. After blocking with 20% AquaBlock(EastCoast) for 30 min at room temperature, cells were incubated overnight at 4° C. with goat anti-insulin antibody (1:100; abcam), rabbit anti-C-peptide antibody (1:100; Cell Signaling), mouse anti-glucagon antibody (1:250; Sigma) or rabbit anti-PDX-1 antiserum (Noguchi et al., 2003) (1:1,000), and then for 1 h at room temperature with FITC-conjugated anti-goat IgG (1:250; Abcam), Alexa Fluor® 647-conjugated anti-rabbit IgG (1:250; Cell Signaling), TRITC conjugated anti-mouse IgG (1:250; Sigma) or FITC-conjugated anti-rabbit IgG (1:100; Jackson Immunochemicals). Mounting medium for fluorescence with DAPI (Vector Laboratories) was used for mounting.

Insulin Release Assay: Insulin release was measured by incubating the cells in Functionality/Viability Medium CMRL1066 (Mediatech). The cells were washed 3 times in PBS and incubated in the solution (Functionality/Viability Medium CMRL1066) with 2.8 mM D-glucose 6 times for each 20 min (total 2 hr) to wash. The cells were then incubated in the solution with 2.8 mM D-glucose for 2 hrs and then the solution with 20 mM D-glucose for 2 hrs. The insulin levels in culture supernatants were measured using Ultra Sensitive Mouse Insulin ELISA (enzyme-linked immunosorbent assay) kit (Mercodia).

Statistics: Data was expressed as mean±SE. Two groups were compared by the Student's t-test. The differences between each group were considered significant if the P value was <0.05.

The inventors have previously reported the establishment of pancreatic stem cell lines from mouse pancreatic tissue of eight-week-old mice without genetic manipulation (Noguchi et al., 2009). The inventors studied the probability of establishment of mouse pancreatic stem cells from donors of several ages without genetic manipulation. The present inventors were able to generate mouse pancreatic stem cells in two of two studies when using new-born mouse pancreata. On the other hand, the inventors were able to generate mouse pancreatic stem cells in only two of twenty studies when using 8-week-old mouse pancreata and were not able to establish stem cells from any of twenty studies when using 24-week-old mouse pancreata (Table 2). This is due to the differences in the number of pancreatic stem cells in each pancreas. There may be some pancreatic stem cells in young pancreata but less or no stem cells in older pancreata. These data suggest that it is difficult to generate mouse pancreatic stem cells from older-donor pancreata without genetic manipulation.

TABLE 2
Efficacy of establishment of mouse pancreatic stem cell lines
without genetic manipulation.

| Differentiation | | | Gene Expression | | | | | | |
|---|---|---|---|---|---|---|---|---|---|
| | suc#/ | | Oct3/ | | | | | | adipo- |
| Old | iso# | PSC# | 4 | Foxa2 | Pdx1 | Ngn3 | β | α | cyte |
| 0 w | 2/2 | #1 | ± | + | + | − | + | + | ND |
| | | #2 | ± | + | + | − | + | + | ND |
| 8 w | 2/20 | #3* | ± | + | + | − | + | + | — |
| | | #4 | ± | + | + | − | + | + | ND |
| 24 w | 0/20 | | | | | | | | |

suc#/iso#: successful isolation number of pancreatic stem cells/total isolation number
PSC: pancreatic stem cells
ND: no data
*One clone in #3 is HN#13 cells

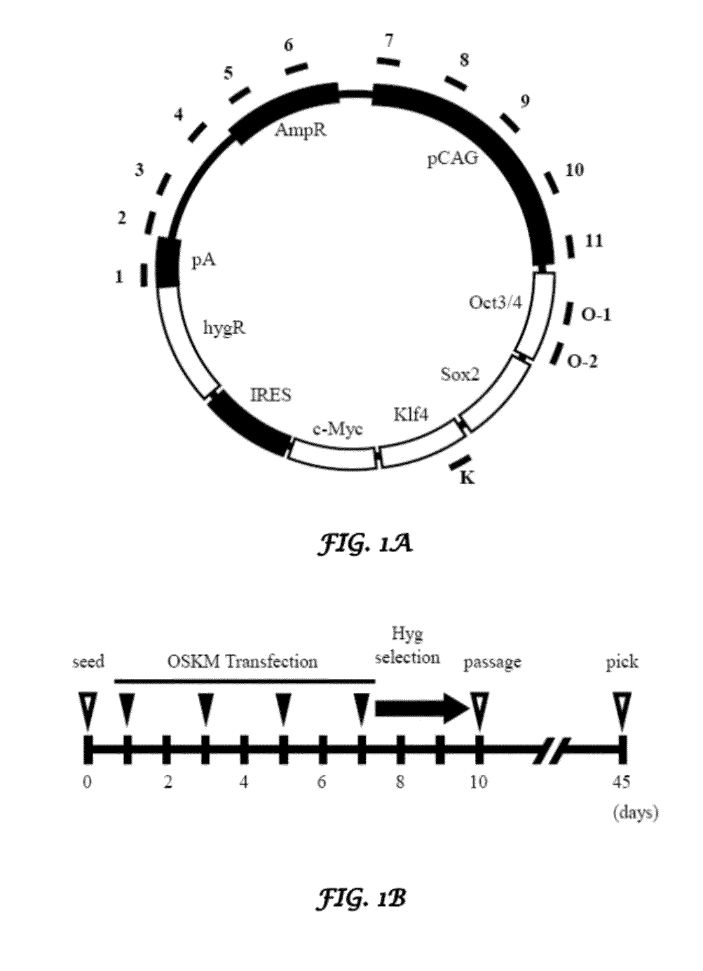

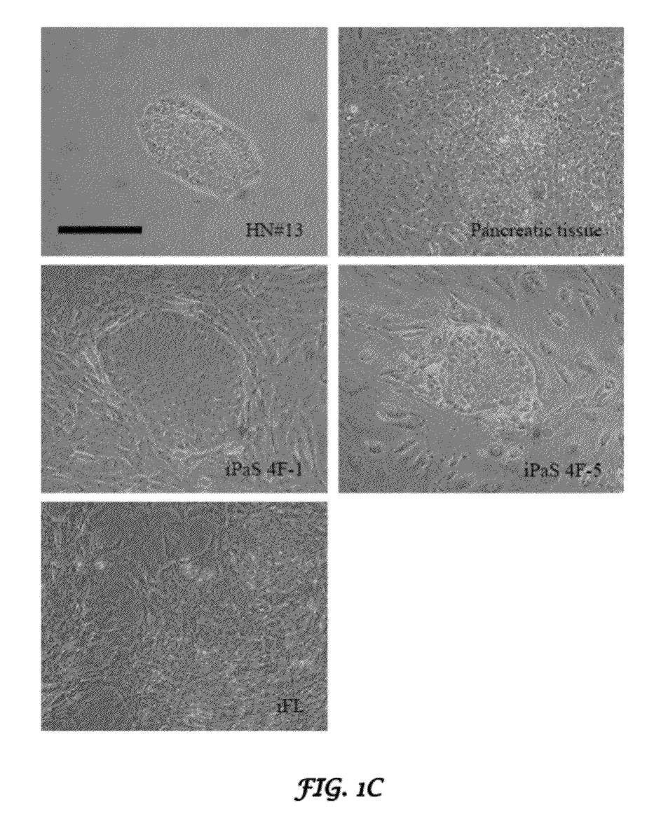

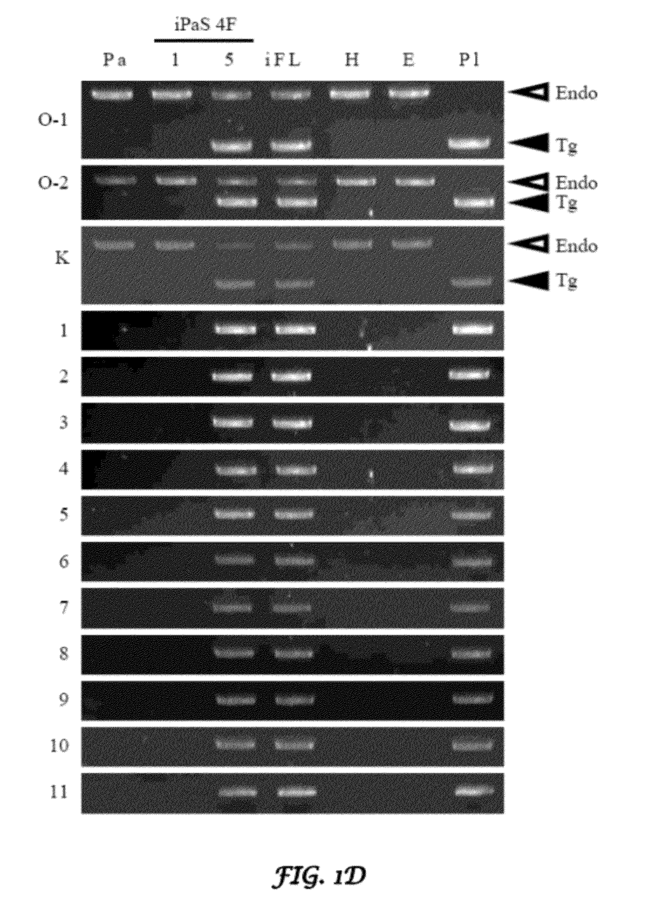

The inventors generated mouse iPS cells from older-donor pancreata by transfection of a single plasmid expressing Oct3/4, Sox2, Klf4 and c-Myc. The four cDNAs encoding Oct3/4, Sox2, Klf4, and c-Myc were connected in this order with the 2A peptide and inserted into a plasmid containing the CAG promoter (Niwa et al., 1991) (FIG. 1A). The inventors transfected the OSKM plasmid into pancreatic tissue from 24-week-old mice on days 1, 3, 5, and 7 (FIG. 1B). The present inventors were unable to generate iPS cells from 24-week-old mouse pancreata. However, it was noticed that there were some cells which had self-renewing capacity potency. The morphology of some cells was similar to that of mouse pancreatic stem cells, which was previously established from young donor pancreata without genetic manipulation. The inventors designated them: induced pancreatic stem (iPaS) cells. The morphology of other cells was similar to that of fibroblast cells, which we designated: induced fibroblast-like (iFL) cells (FIG. 1C).

To evaluate the plasmid integration in these cells, genomic DNA was amplified by polymerase chain reaction (PCR) with primers (FIG. 1A, Table 1). Although PCR detected plasmid incorporation into the host genome of some cells, no amplification of plasmid DNA was observed in several cells, such as iPaS 4F-1 (FIG. 1D). Although one cannot formally exclude the presence of small plasmid fragments, these data show that some of the cells that have self-renewal capacity are most likely free from plasmid integration into the host genome.

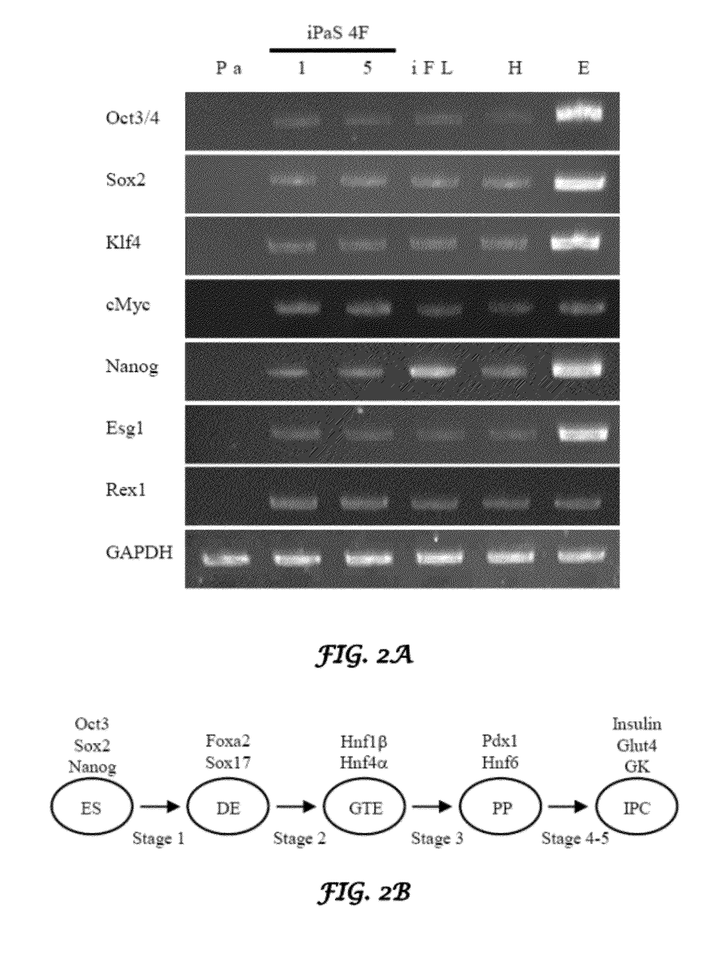

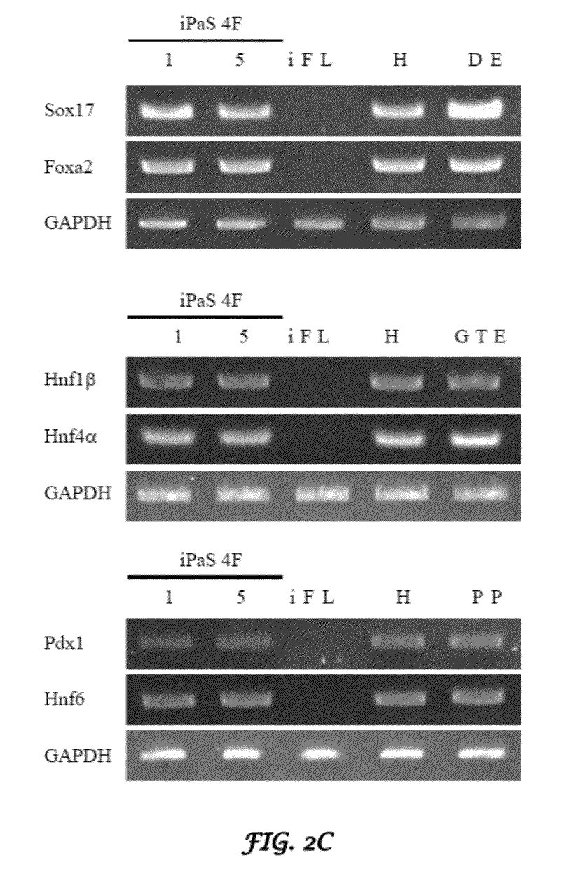

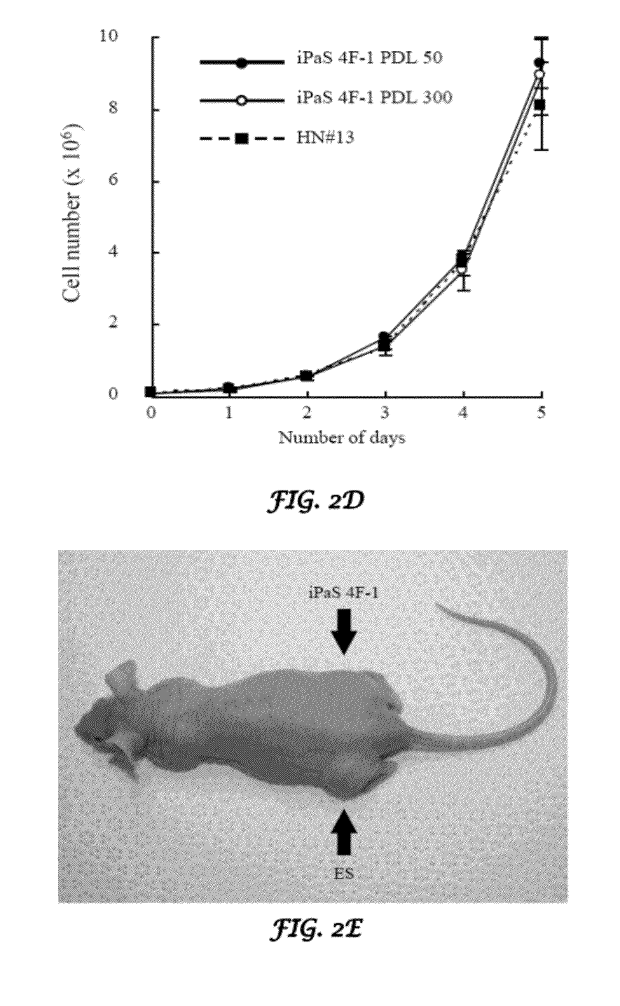

To study gene expression in these cells, reverse transcription PCR(RT-PCR) analysis of ES cell marker genes was performed. RT-PCR revealed that both pancreatic stem cell-like clones and fibroblast-like clones expressed some ES cell markers, including Oct3/4, Sox2, Klf4, c-Myc, Nanog, Esg1, Ecat, and Rex1. However, the expression levels seemed to be lower than in ES cells (FIG. 2A). The inventors also studied gene expression patterns of endodermal/pancreatic progenitor cell markers. Differentiated cells from ES cells (generated by a stepwise differentiation protocol that relies on intermediates thought to be similar to cell populations present in the developing embryo) (D'Amour et al., 2006; Kroon et al., 2008) were used as a positive control (FIG. 2B). The marker gene expression patterns of the definitive endoderm (sex determining region Y-box17; Sox17, forkhead box protein a2; Foxa2), gut tube endoderm (hepatocyte nuclear factor 1β; Hnf1β, Hnf4α), and pancreatic progenitors (Hnf6, Pdx1) were detected in iPaS cells, which is similar to patterns in the mouse pancreatic stem cell line, HN#13, but not iFL cells (FIG. 2C). The iPaS 4F-1 cells continue to divide actively beyond the population doubling level (PDL) 300 without changes in morphology or growth activity (FIG. 2D). To examine teratoma formation and tumorigenic potential in vivo, iPaS 4F-1 cells (1×10⁷) at PDL 150 were transplanted into nude mice. No teratoma/tumors developed in the nude mice that received iPaS 4F-1 cells at during an observation period of at least six months, as is the case with HN#13 cells (Noguchi et al., 2009). In contrast, sites injected with 1×10⁷ ES cells developed teratoma about three weeks after transplantation (FIG. 2E). These data indicate that the endodermal marker expression pattern of iPaS cells is similar to the mouse pancreatic stem cell line, HN#13 used herein, but is different than the expression pattern of ES cells.

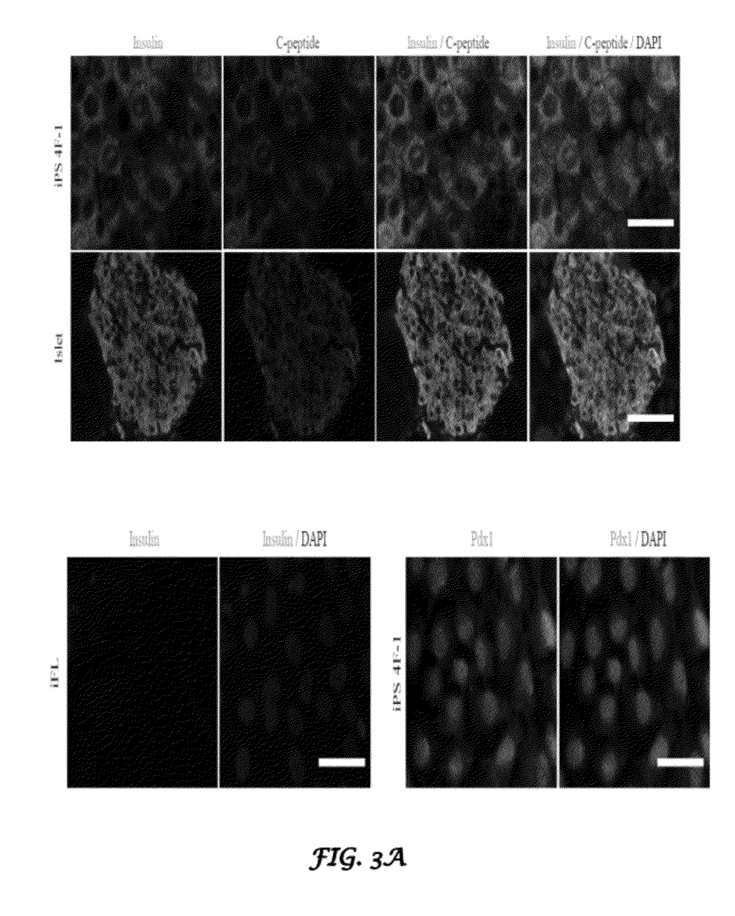

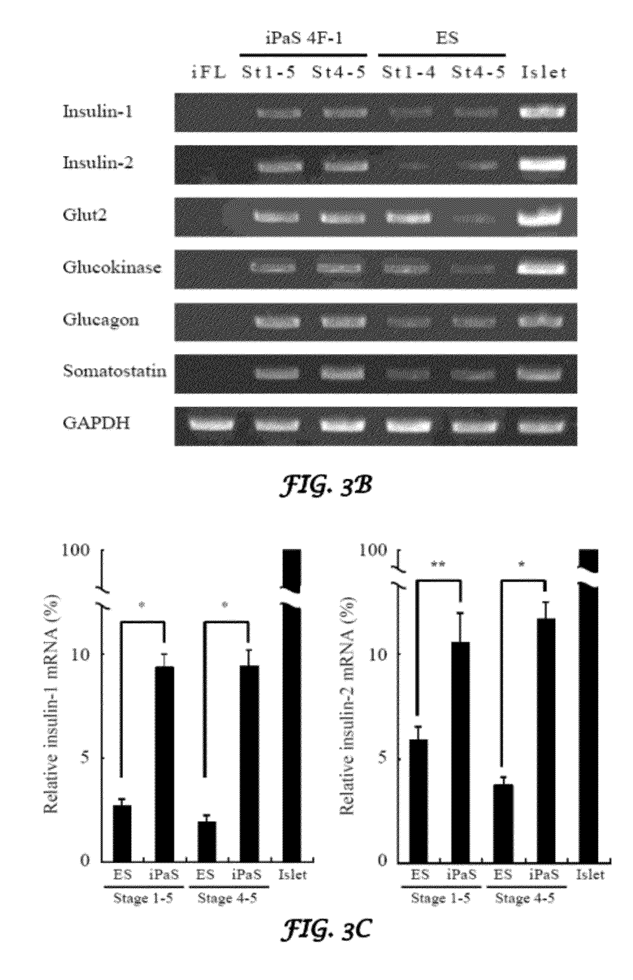

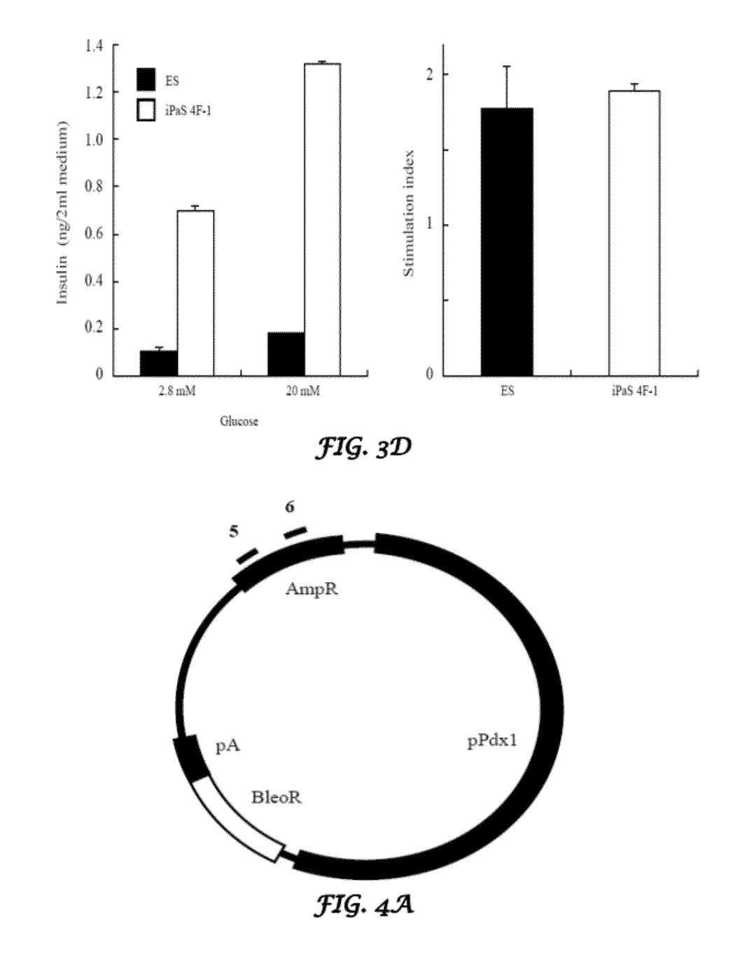

To determine whether iPaS cells can be differentiated into insulin-producing cells, the inventors applied the stepwise differentiation protocol shown in FIG. 2B. The stepwise differentiation protocol relies on intermediates thought to be similar to cell populations present in the developing embryo (D'Amour et al., 2006; Kroon et al., 2008). ES cells differentiate into definitive endoderm (DE) in stage 1; DE cells differentiate into gut tube endoderm (GTE) in stage 2; GTE cells differentiate into pancreatic progenitors (PP) in stage 3; and PP cells differentiate into insulin-producing cells (IPC) in stages 4 and 5. Since iPaS 4F-1 cells express endodermal cell markers (PP cell markers), the present inventors also included stages 4 and 5 of the induction protocol in the stepwise differentiation protocol. Differentiated cells from ES cells (generated by the stepwise differentiation protocol (Stage 1-5) or the stage 4-5 protocol) were used as a control. The iPaS 4F-1 cells were differentiated into insulin-producing cells (FIG. 3A) more efficiently than ES cells by both the stepwise differentiation protocol and the stage 4-5 protocol (FIGS. 3B and 3C). Insulin-positive cells were C-peptide positive, thus excluding insulin uptake from the media. The iFL cells were unable to be differentiated into insulin-producing cells (FIG. 3A). RT-PCR analysis confirmed the expression of endocrine-specific gene products of insulin-1 and -2, Glut2, glucokinase, glucagon, and somatostatin (FIG. 3B). To evaluate whether the differentiated cells have glucose sensitivity, the differentiated cells from iPaS 4F-1 cells were exposed to low (2.8 mM) or high (20 mM) concentrations of glucose. The cells released about 6-fold higher amounts of mouse insulin than an ES-derived population on both glucose concentrations (FIG. 3D). The stimulation index was similar between the differentiated cells from iPaS 4F-1 cells and ES cells.

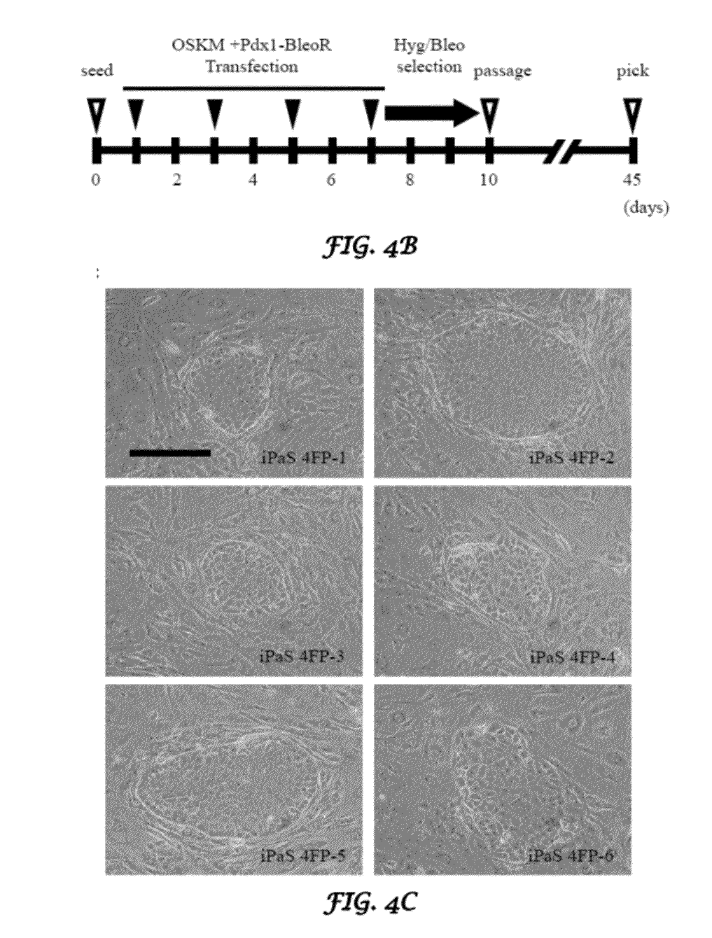

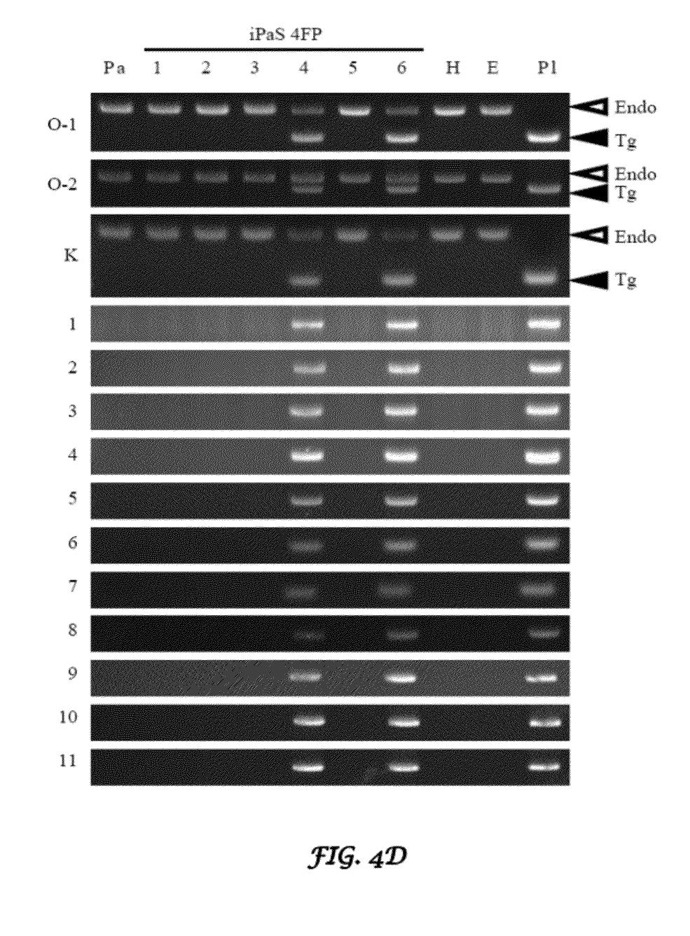

The present inventors attempted efficient selection of iPaS cells, since there were a large number of iFL cells in the first study. Since iPaS 4F-1 cells expressed Pdx1 transcription factor at both the mRNA (FIG. 2C) and protein level (FIG. 3A), the inventors used a plasmid containing a bleomycin-resistance (BleoR) gene that was driven by the Pdx1 promoter (FIG. 4A). The inventors transfected the OSKM plasmid and the Pdx1-BleoR plasmid together in pancreatic tissue from a 24-week-old mouse on days 1, 3, 5, and 7 (FIG. 4B) and obtained multiple colonies (iPaS 4FP-1 to 6) that had self-renewal capacity and were morphologically similar to iPaS 4F-1 cells. The morphology of iPaS 4FP-1 to 6 cells is shown in FIG. 4C. There were few fibroblast-like colonies in this study. To evaluate the plasmid integration in these cells, genomic DNA from these cells was amplified by PCR with primers indicated in FIG. 1A. Although PCR detected plasmid incorporation into the host genome of some cells, no amplification of plasmid DNA was observed in iPaS 4FP-1, -2, -3, and -5 cells (FIG. 4D). Although it is not possible to formally exclude the presence of small plasmid fragments, these data show that these cells are most likely free of plasmid integration into the host genome.

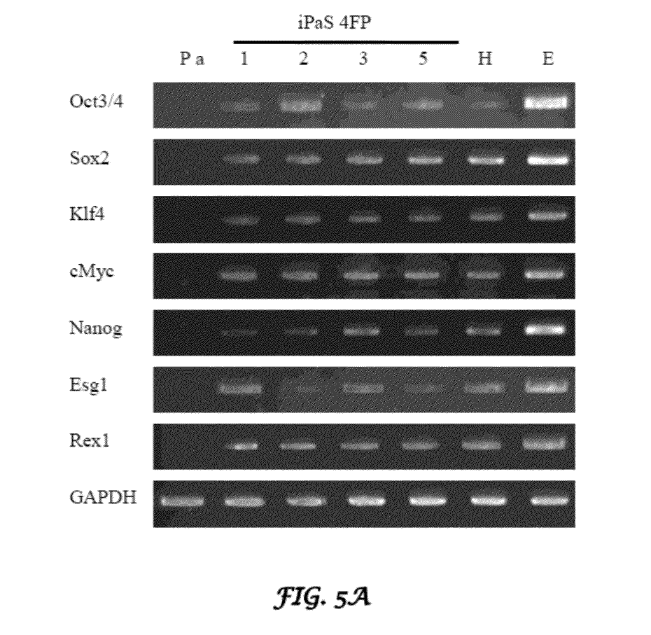

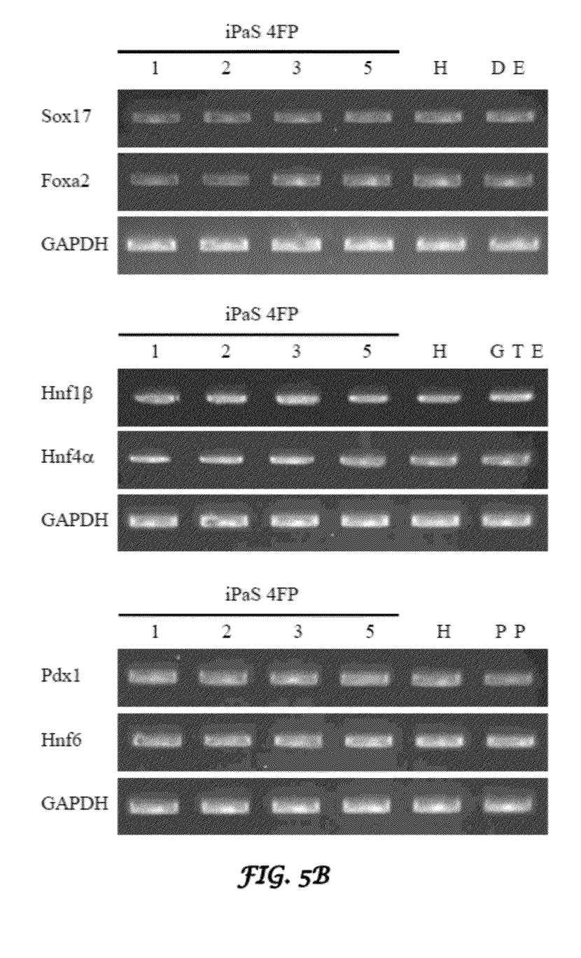

To study the gene expression profile in these cells, RT-PCR analysis of ES cell marker genes and endodermal marker genes was performed. Although RT-PCR revealed that these iPaS 4FP colonies expressed some ES cell markers, expression levels seemed to be lower than in ES cells (FIG. 5A). The marker genes of the definitive endoderm, gut tube endoderm, and pancreatic progenitors were detected in all iPaS 4F cells (FIG. 5B). To examine teratoma formation and tumorigenic potential in vivo, iPaS 4FP-1, -2, -3, and -5 cells (1×10⁷) at PDL 150 were transplanted into nude mice. No teratoma/tumors developed in the nude mice receiving all of iPaS 4FP cells at either stage during an observation period of at least six months (FIG. 5C). These data indicate that the iPaS 4FP cells express endodermal markers, similar to HN#13 and iPaS 4F-1 cells.

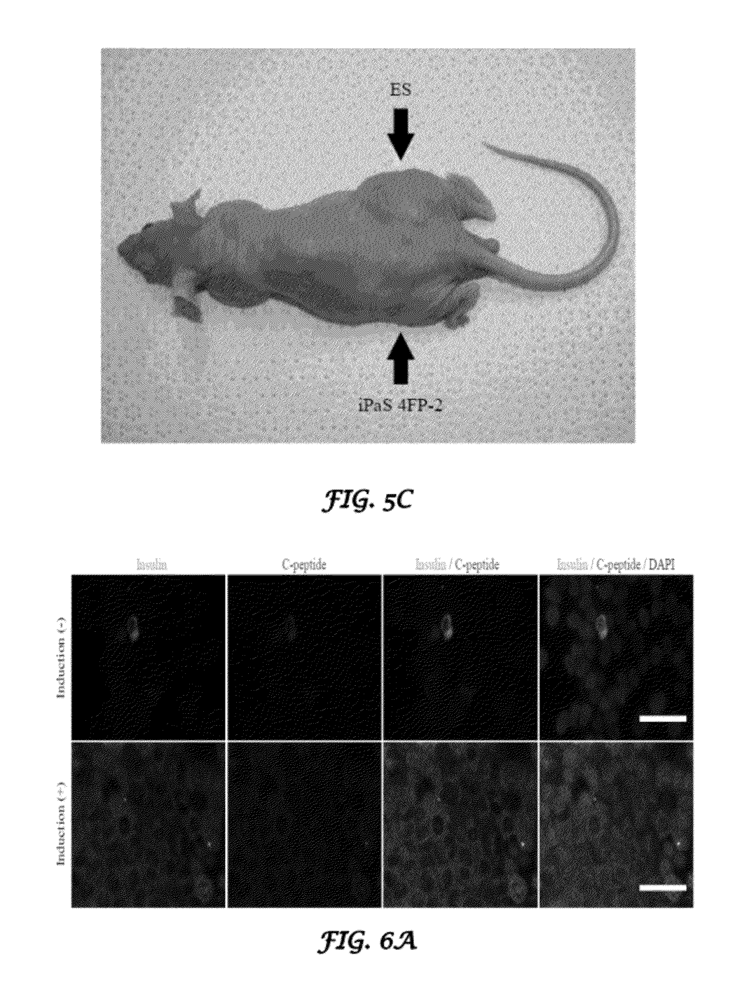

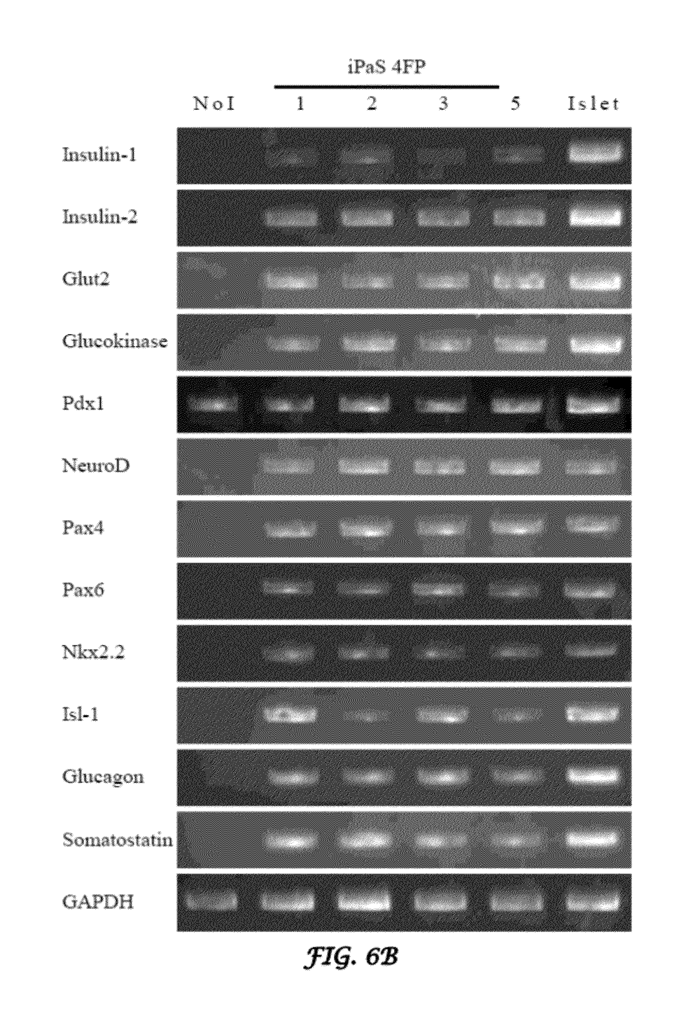

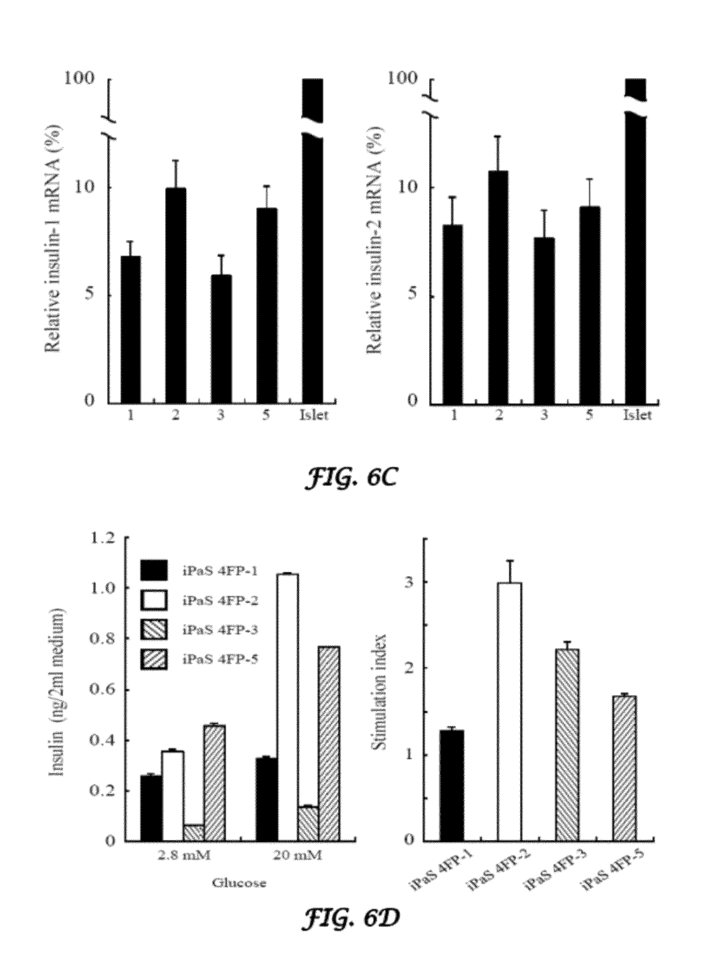

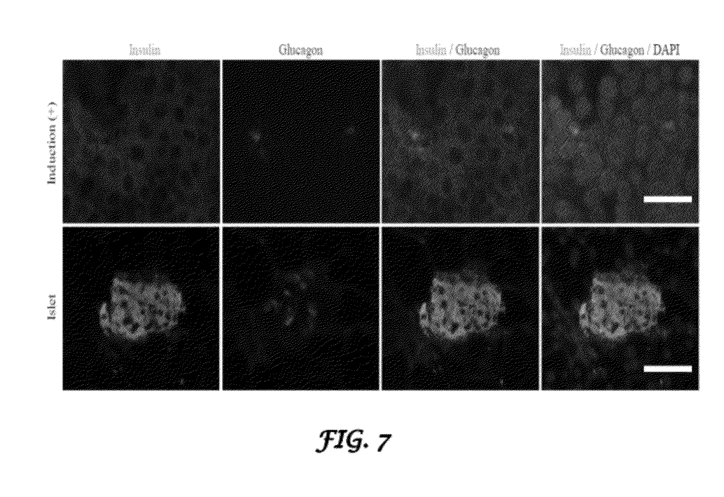

To determine the ability of the generated cells to differentiate into insulin-producing cells, the inventors applied the stage 4-5 protocol from the stepwise differentiation protocol (shown in FIG. 2B). All of the iPaS 4FP clones without plasmid integration were differentiated into insulin-producing cells by the stage 4-5 protocol (FIG. 6A-6C). Insulin-positive cells were C-peptide positive, excluding insulin uptake from the media. Some of cells were also positive for glucagon (FIG. 7). RT-PCR analysis confirmed the expression of endocrine-specific gene products of insulin-1 and -2, Glut2, glucokinase, NeuroD, Pax4, Pax6, Nkx2.2, Isl-1, glucagon, and somatostatin (FIG. 6B). To evaluate whether the differentiated cells have glucose sensitivity, the differentiated cells from iPaS 4FP-1, -2, -3, and -5 cells were exposed to low or high concentrations of glucose. All of these clones released mouse insulin at both low and high glucose (FIG. 6D), although the amount of insulin was different among them. The stimulation index was also different among the clones. These data suggest that the Pdx1-BleoR plasmid can efficiently select iPaS cells, but the differentiation ability of the cells into insulin-producing cells depends on each clone.

The iPS technology described herein has significant implications for overcoming most of the ethical issues associate with ES cell derivation from embryos. However, the iPS cells still have some ethical issues because they have similar or the same potency as ES cells. To focus on the treatment of diabetic patients, differentiated tissue is needed that includes insulin-producing cells. Although islet transplantation is one of the efficient strategies for the treatment of diabetes (Shapiro 2000), it is circumscribed by the limited and irregular supply of cadaveric donors and the risks of immunosuppressant therapy. In this study, the inventors induced pancreatic stem cells from mouse pancreatic tissue by transient overexpression of reprogramming factors and Pdx1 selection. The iPaS cells were able to differentiate into insulin-producing cells more efficiently than ES cells. On the other hand, the iPaS cells hardly differentiated adipocytes or osteocytes (data not shown). Since the iPaS cells are pancreas-specific stem cells, the use of these cells seems to have less ethical concerns than ES cells and even iPS cells. Moreover, the iPaS cells have no teratoma formation. This is one of the advantages of iPaS cells on clinical application compared with iPS cells. iPS cells have a risk for teratoma formation, even after transplantation of differentiated cells derived from iPS cells due to contamination of undifferentiated cells.

Insulin-producing cells derived from iPaS cells expressed 2- to 5-fold higher insulin mRNA and about 6-fold higher insulin production compared with those derived from ES cells. Insulin-producing cells derived from iPaS cells are also glucose responsive. Moreover, iPaS cells do not need to be treated with stages 1 to 3 of the stepwise differentiation protocol to differentiate into insulin-producing cells. These are also advantages of iPaS cells compared with ES cells and, probably, iPS cells. However, insulin expression by iPaS cells is at much lower levels compared to insulin expression by pancreatic islets. Although the present inventors transplanted 1×10⁸ insulin-producing cells derived from iPaS cells into syngeneic diabetic mice, the blood glucose levels of none of the 5 mice receiving the cells reached normoglycemia. Further optimization of the conditions (stages 4 and 5) is needed to generate a sufficient yield of insulin-producing cells for transplantation to treat diabetes.

Interestingly, the inventors observed differences between iPaS lines from the same donor, especially on differentiation ability. The differences between human iPS lines from the same type 1 diabetes patient in the expression of retroviruses expressing reprogramming 4 factors have been reported, potentially due to transgene reactivation or incomplete silencing (Maehr et al., 2009). Since the iPaS 4FP-1, -2, -3, and -5 cells of the present invention seem to have no plasmid integration into the host DNA, the differences between iPaS lines from the same donor may be due to other reasons rather than gene integration.

Some groups have shown that overexpression of Pdx1, Ngn3, NeuroD, and/or MafA by adenoviruses in vivo directly converted liver cells (Ferber et al., 2000; Kaneto et al., 2005a; Kaneto et al., 2005b) or pancreatic tissue (Zhou et al., 2008) into insulin-producing cells, suggesting a direct reprogramming without reversion to a pluripotent stem cell state. More recently, direct conversion of fibroblasts to functional neurons by Ascl1, Brn2 (also called Pou3f2) and Myt11 (Vierbuchen et al., 2010) was reported. These reports of direct reprogramming without reversion to a pluripotent stem cell state seem to have lower ethical issues than iPS cells and, therefore, could have important implications for studies of cell differentiation and regenerative medicine. However, these strategies require a large number of mature cells and the induction therapy has to be done on all of these cells directly because they are not stem cells and do not have self-renewal capacity. Two major advantages of iPS/iPaS cells are that they can be generated from small amount of cells and they will expand to enough cells because they have self-renewal capacity.

The present invention generates iPaS cells from mouse pancreatic tissue by transient overexpression of reprogramming factors and Pdx1 selection. Generation of iPaS cells and the differentiation into insulin-producing cells are relevant for the possibility of autologous cell replacement therapy, probably more efficiently than iPS cells. The technology to generate iPaS cells by reprogramming factors and tissue-specific selection may also be useful for the generation of other tissue-specific stem cells.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCES

U.S. Patent Application Publication No. 2008/0233649: Pancreatic Stem Cells.

U.S. Patent Application Publication No. 2010/0137202: Compositions for Reprogramming A Cell and Uses Therefor.

Bonner-Weir, S., Taneja, M., Weir, G. C., Tatarkiewicz, K., Song, K. H., Sharma, A., and O'Neil, J. J. (2000). In Vitro Cultivation of Human Islets from Expanded Ductal Tissue. Proc. Natl. Acad. Sci. USA. 97, 7999-8004.

D'Amour, K. A., Bang, A. G., Eliazer, S., Kelly, O. G., Agulnick, A. D., Smart, N. G., Moorman, M. A., Kroon, E., Carpenter, M. K., and Baetge, E. E. (2006). Production of Pancreatic Hormone-Expressing Endocrine Cells From Human Embryonic Stem Cells. Nat. Biotechnol. 24, 1392-1401.

Ferber, S., Halkin, A., Cohen, H., Ber, I., Einav, Y., Goldberg, I., Barshack, I., Seijffers, R., Kopolovic, J., Kaiser, N., and Karasik, A. (2000). Pancreatic and Duodenal Homeobox Gene 1 Induces Expression of Insulin Genes in Liver and Ameliorates Streptozotocin-Induced Hyperglycemia. Nat. Med. 6, 568-572.

Gao, R., Ustinov, J., Pulkkinen, M. A., Lundin, K., Korsgren, O., and Otonkoski, T. (2003). Characterization of Endocrine Progenitor Cells and Critical Factors for Their Differentiation in Human Adult Pancreatic Cell Culture. Diabetes 52, 2007-1015.

Heremans, Y., Van De Casteele, M., in't Veld, P., Gradwohl, G., Serup, P., Madsen, O., Pipeleers, D., and Heimberg, H. (2002). Recapitulation of Embryonic Neuroendocrine Differentiation in Adult Human Pancreatic Duct Cells Expressing Neurogenin 3. J. Cell Biol. 159, 303-312.

Kaneto, H., Matsuoka, T. A., Nakatani, Y., Miyatsuka, T., Matsuhisa, M., Hori, M., and Yamasaki, Y (2005a). A Crucial Role of Mafa as A Novel Therapeutic Target for Diabetes. J. Biol. Chem. 280, 15047-15052.

Kaneto, H., Nakatani, Y., Miyatsuka, T., Matsuoka, T. A., Matsuhisa, M., Hori, M., and Yamasaki, Y. (2005b). PDX-1/VP16 Fusion Protein, Together with Neurod or Ngn3, Markedly Induces Insulin Gene Transcription and Ameliorates Glucose Tolerance. Diabetes 54, 1009-1022.

Kroon, E., Martinson, L. A., Kadoya, K., Bang, A. G., Kelly, O. G., Eliazer, S., Young, H., Richardson, M., Smart, N. G., Cunningham, J., Agulnick, A. D., D'Amour, K. A., Carpenter, M. K., and Baetge, E. E. (2008). Pancreatic Endoderm Derived From Human Embryonic Stem Cells Generates Glucose-Responsive Insulin-Secreting Cells In Vivo. Nat. Biotechnol. 26, 443-452.

Lowry, W. E., Richter, L., Yachechko, R., Pyle, A. D., Tchieu, J., Sridharan, R., Clark, A. T., and Plath, K. (2008). Generation of Human Induced Pluripotent Stem Cells from Dermal Fibroblasts. Proc. Natl. Acad. Sci. USA 105, 2883-2888.

Maehr, R., Chen, S., Snitow, M., Ludwig, T., Yagasaki, L., Goland, R., Leibel, R. L., Melton, D. A. (2009). Generation of Pluripotent Stem Cells From Patients With Type 1 Diabetes. Proc. Natl. Acad. Sci. USA 106, 15768-15773.

Maherali, N., Sridharan, R., Xie, W., Utikal, J., Eminli, S., Arnold, K., Stadtfeld, M., Yachechko, R., Tchieu, J., Jaenisch, R., Plath, K., and Hochedlinger, K. (2007). Directly Reprogrammed Fibroblasts Show Global Epigenetic Remodeling and Widespread Tissue Contribution. Cell Stem Cell 1, 55-70.

Niwa, H., Yamamura, K., and Miyazaki, J. (1991). Efficient Selection for High-Expression Transfectants with A Novel Eukaryotic Vector. Gene. 108, 193-199.

Noguchi, H., Kobayashi, N., Westerman, K. A., Sakaguchi, M., Okitsu, T., Totsugawa, T., Watanabe, T., Matsumura, T., Fujiwara, T., Ueda, T., Miyazaki, M., Tanaka, N., and Leboulch, P. (2002). Controlled Expansion of Human Endothelial Cell Populations by Cre-1oxp-based Reversible Immortalization. Hum. Gene Ther. 13, 321-334.

Noguchi, H., Kaneto, H., Weir, G. C., Bonner-Weir, S. (2003). PDX-1 Protein Containing its own Antennapedia-Like Protein Transduction Domain can Transduce Pancreatic Duct and Islet Cells. Diabetes. 52, 1732-1737.

Noguchi, H., Oishi, K., Ueda, M., Yukawa, H., Hayashi, S., Kobayashi, N., Levy, M. F., and Matusmoto, S. (2009). Establishment of Mouse Pancreatic Stem Cell Line. Cell Transplant. 18,563-571.

Okita, K., Ichisaka, T., and Yamanaka S. (2007). Generation of Germline-Competent Induced Pluripotent Stem Cells. Nature. 448, 313-317.

Okita, K., Nakagawa, M., Hyenjong, H., Ichisaka, T., and Yamanaka S. (2008). Generation of Mouse Induced Pluripotent Stem Cells Without Viral Vectors. Science. 322, 949-953.

Park, I. H., Zhao, R., West, J. A., Yabuuchi, A., Huo, H., Ince, T. A., Lerou, P. H., Lensch, M. W., Daley, G. Q. (2008). Reprogramming of Human Somatic Cells to Pluripotency with Defined Factors. Nature. 451, 141-146.

Shapiro, A. M., Lakey, J. R., Ryan, E. A., Korbutt, G. S., Toth, E., Warnock, G. L., Kneteman, N. M., and Rajotte, R. V. (2000). Islet Transplantation in Seven Patients with Type 1 Diabetes Mellitus using A Glucocorticoid-Free Immunosuppressive Regimen. N. Engl. J. Med. 343, 230-238.

Stadtfeld, M., Nagaya, M., Utikal, J., Weir, G., and Hochedlinger, K. (2008). Induced Pluripotent Stem Cells Generated without Viral Integration. Science. 322, 945-949.

Street, C. N., Lakey, J. R., Shapiro, A. M., Imes, S., Rajotte, R. V., Ryan, E. A., Lyon, J. G., Kin, T., Avila, J., Tsujimura, T., and Korbutt, G. S. (2004). Islet Graft Assessment in the Edmonton Protocol: Implications for Predicting Long-Term Clinical Outcome. Diabetes. 53, 3107-3114.

Takahashi, K., and Yamanaka S. (2006). Induction of Pluripotent Stem Cells from Mouse Embryonic and Adult Fibroblast Cultures by Defined Factors. Cell. 126, 663-676.

Takahashi, K., Tanabe, K., Ohnuki, M., Narita, M., Ichisaka, T., Tomoda, K., and Yamanaka S. (2007). Induction of Pluripotent Stem Cells from Adult Human Fibroblasts by Defined Factors. Cell. 131, 861-872.

Vierbuchen, T., Ostermeier, A., Pang, Z. P., Kokubu, Y., Südhof, T. C., and Wernig, M. (2010). Direct Conversion of Fibroblasts to Functional Neurons by Defined Factors. Nature. 463, 1035-1041.

Wernig, M., Meissner, A., Foreman, R., Brambrink, T., Ku, M., Hochedlinger, K., Bernstein, B. E., and Jaenisch, R. (2007). In Vitro Reprogramming of Fibroblasts into A Pluripotent ES-Cell-Like State. Nature. 448, 318-324.

Yamamoto, T., Yamato, E., Taniguchi, H., Shimoda, M., Tashiro, F., Hosoi, M., Sato, T., Fujii, S., and Miyazaki, J. I. (2006). Stimulation of Camp Signalling Allows Isolation of Clonal Pancreatic Precursor Cells from Adult Mouse Pancreas. Diabetologia. 49, 2359-2367.

Yu, J., Vodyanik, M. A., Smuga-Otto, K., Antosiewicz-Bourget, J., Frane, J. L., Tian, S., Nie, J., Jonsdottir, G. A., Ruotti, V., Stewart, R., Slukvin, I. I., Thomson, J. A. (2007). Induced Pluripotent Stem Cell Lines Derived from Human Somatic Cells. Science. 318, 1917-1920.

Yu, J., Hu, K., Smuga-Otto, K., Tian, S., Stewart, R., Slukvin, I. I., and Thomson, J. A. (2009). Human Induced Pluripotent Stem Cells Free of Vector and Transgene Sequences. Science. 324, 797-801.

Zhou, Q., Brown, J., Kanarek, A., Rajagopal, J., Melton, D. A. (2008). In Vivo Reprogramming of Adult Pancreatic Exocrine Cells to Beta-Cells. Nature. 455, 627-632.

What is claimed is:

1. A composition for islet transplantation comprising one or more induced pancreatic stem (iPaS) cells, wherein the iPaS are obtained from differentiated pancreatic ductal cells that are modified into one or more insulin-producing cells by the expression of one or more transcription factors and by an expression of one or more genes selected from the group consisting of Oct3/4, Sox2, Klf4, and c-Myc.

2. The composition of claim 1, wherein the transcription factor is Pdx1.

3. The composition of claim 1, wherein the iPaS cells are generated from a pancreatic tissue of a donor.

4. The composition of claim 3, wherein the donor is a human donor, a mouse, a primate, or any other vertebrate species.

5. The composition of claim 1, wherein the composition is used for the treatment of diabetes.

6. A method for generating one or more induced pancreatic stem (iPaS) cells from a pancreatic tissue of a vertebrate donor comprising the steps of:
digesting the pancreatic tissue from the vertebrate donor;
removing one or more fibroblast cells from the digested tissue cells;
culturing the digested tissue cells without the fibroblast cells in a growth medium;
transfecting the cultured cells with a first plasmid encoding one or more cell marker genes and a promoter, wherein the cell marker genes are selected from the group consisting of Oct3/4, Sox2, Klf4, and c-Myc;
transfecting the cultured cells with a second plasmid encoding one or more transcription factors, wherein the transcription factor comprises Pdx1; and
harvesting one or more colonies of iPaS cells following the transfection of the first and the second plasmid.

7. The method of claim 6, further comprising the steps of:
performing a polymerase chain reaction (PCR) analysis on the transfected cells to determine a plasmid integration and an expression of the one or more cell marker genes; and
performing an immunoassay or any other suitable assay to determine a level of insulin produced by the generated iPaS cells.

8. An induced pancreatic stem (iPaS) cell made by the method of claim 6.

9. A method of treating diabetes in a patient comprising the steps of:
identifying the patient in need of treatment against the diabetes;
infusing a therapeutically effective amount of an islet transplantation composition into a liver of the patient through a catheter, wherein the islet transplantation composition comprises one or more induced pancreatic stem (iPaS) cells; and
administering an optional immunosuppressant to the patient to prevent a rejection of the one or more infused islets.

10. The method of claim 9, wherein the iPaS differentiates into one or more insulin-producing cells under an influence of one or more transcription factors.

11. The method of claim 10, wherein the transcription factor is Pdx1.

12. The method of claim 9, wherein the iPaS cells expresses one or more cell markers selected from the group consisting of Oct3/4, Sox2, Klf4, and c-Myc.

13. The method of claim 9, wherein the iPaS cells are generated from a pancreatic tissue of a donor.

14. The method of claim 13, wherein the donor is a human donor, a mouse, a primate, or any other vertebrate species.

15. The method of claim 9, further comprising the step of measuring a glucose level, an insulin level, or both in the patient at one or more definite intervals post transplantation.

16. An induced pluripotent stem (iPS) cell colony, wherein the iPS cell colony is made from a tissue of a donor by transfection with one or more plasmids encoding one or more transcription factors, cell marker genes, or both.

17. The iPS cell colony of claim 16, wherein the donor comprises a human donor, a mouse, a primate or any other vertebrate species.

18. The iPS cell colony of claim 16, wherein the tissue comprises a pancreatic tissue, a kidney tissue, a liver tissue, a heart tissue, or a splenic tissue.

19. A method for generating one or more induced pluripotent stem (iPS) cells ex vivo from a pancreatic tissue of a donor comprising the steps of:
digesting the donor tissue;
culturing the digested tissue cells in a growth medium;
transfecting the cultured cells with one or more plasmids encoding one or more cell marker genes and a promoter, a transcription factor or both; and
harvesting one or more colonies of iPS cells following the transfection of the plasmid.

20. The method of claim 19, further comprising the steps of:
performing an optional step of removing one or more fibroblast cells from the digested tissue cells; and
performing a PCR analysis of the transfected cells to determine a plasmid integration and an expression of the one or more cell marker genes

21. The method of claim 19, wherein the donor comprises a human donor, a mouse, a primate, or any other vertebrate species.

22. The method of claim 19, wherein the tissue comprises a pancreatic tissue, a kidney tissue, a liver tissue, a heart tissue, or a splenic tissue.

23. The method of claim 19, wherein the tissue is a pancreatic tissue.

24. The method of claim 19, wherein the cell marker genes are selected from the group consisting of Oct3/4, Sox2, Klf4, and c-Myc and the transcription factor is Pdx1.

25. An induced pluripotent stem (iPS) cell generated by the method of claim 19.
